(19) United States
(12) Patent Application Publication
(10) Pub. No.: US 2025/0339517 A1
(43) Pub. Date: Nov. 6, 2025

(54) LIPID NANOCARRIER VACCINE

(71) Applicant: ROYAL MELBOURNE INSTITUTE OF TECHNOLOGY, Melbourne, Victoria (AU)

(72) Inventors: Sampa SARKAR, Melbourne, Victoria (AU); Sarvesh Kumar SONI, Melbourne, Victoria (AU); Charlotte Elizabeth CONN, Melbourne, Victoria (AU); Calum DRUMMOND, Melbourne, Victoria (AU)

(21) Appl. No.: 18/869,179

(22) PCT Filed: May 26, 2023
(86) PCT No.: PCT/AU2023/050449

§ 371 Date: Nov. 25, 2024

(30) Foreign Application Priority Data
May 26, 2022 (AU) 2022901426

(51) Int. Cl. A61K 39/385; A61K 9/00; A61K 39/00; A61K 39/04; A61P 31/00

(52) U.S. Cl. CPC A61K 39/385; A61K 9/0019; A61K 39/04; A61P 31/00; A61K 2039/54; A61K 2039/55555; A61K 2039/6018; A61K 2039/6087

(57) ABSTRACT

The present disclosure relates to a lipid nanoparticle which is a carrier for an antigen. The present disclosure also relates to an immunogenic composition comprising the antigen. The immunogenic composition may be a vaccine composition. The present disclosure further relates to methods and uses of the carrier and immunogenic composition.

### TECHNICAL FIELD

The present disclosure relates to a lipid nanoparticle which is a carrier for an antigen, and uses thereof.

### BACKGROUND

Vaccine delivery is a broad field of research on the development of novel materials or carrier systems for effective therapeutic delivery of antigens. Carrier systems for subunit vaccines are required to overcome various challenges relating to the nature of the antigen being delivered including, but not limited to, poor solubility, low bioavailability, reduced half-life, lack of selectivity, poor cell interactions, and toxicity.

Many different delivery approaches have been tested including polymeric delivery vehicles, conjugates, and core shell particles. A suitable delivery approach must be able to circulate systemically for an appropriate time, be capable of protecting the antigen during this time, and be adapted for a suitable fusion or other delivery event into the cell of interest.

Tuberculosis (TB), a communicable disease caused by the pathogen Mycobacterium tuberculosis (MTB), remains one of the top ten causes of death globally, leading to an estimated 1.2 million deaths in 2019. The increasing emergence of drug-resistant TB and slow development of new therapeutics is anticipated to lead to increased global TB-related morbidity and mortality.

The current vaccine of choice, Mycobacterium bovis Bacillus Calmette-Guérin (BCG), was introduced nearly 100 years ago and is the most widely used vaccine in the world. However, while the BCG vaccine efficiently protects against severe disseminated forms of TB in children, it does not prevent the pulmonary disease which is a major cause of TB mortality. One of the major reasons for BCG failure is its inability to produce lung specific immunity and even the systemic immunity diminishes during adolescence.

There is a need to provide carriers for delivery of antigenic agents which can result in generation of a suitable immune response as part of a vaccine composition.

Any reference to background art herein is not to be construed as an admission that such art constitutes common general knowledge in Australia or elsewhere.

### SUMMARY

According to a first aspect of the present disclosure, there is provided a non-lamellar lyotropic liquid crystalline phase carrier comprising one or more lipids forming the carrier and an antigen associated with the carrier.

In a second aspect of the present disclosure, there is provided an immunogenic composition comprising a non-lamellar lyotropic liquid crystalline phase carrier of the first aspect and a pharmaceutically acceptable carrier, diluent and/or excipient.

In embodiments, the immunogenic composition is a vaccine.

In certain examples, the immunogenic composition does not comprise or is substantially free of an adjuvant which is not a constituent part of the non-lamellar lyotropic liquid crystalline phase carrier itself.

In a third aspect of the present disclosure, there is provided a method of delivering an antigen to a cell including the step of contacting the cell with the non-lamellar lyotropic liquid crystalline phase carrier of the first aspect, or the immunogenic composition of the second aspect.

In a fourth aspect of the present disclosure, there is provided a method of inducing an immune response in a subject including the step of administering an effective amount of the non-lamellar lyotropic liquid crystalline phase carrier of the first aspect, or the immunogenic composition of the second aspect, to the subject.

In a fifth aspect of the present disclosure, there is provided a method of preventing, treating or ameliorating an infection, disease, disorder or condition including the step of administering a therapeutically effective amount of a non-lamellar lyotropic liquid crystalline phase carrier of the first aspect, or the immunogenic composition of the second aspect, to a subject in need thereof to thereby prevent, treat or ameliorate the infection, disease, disorder or condition.

In a sixth aspect of the present disclosure, there is provided a non-lamellar lyotropic liquid crystalline phase carrier of the first aspect, or the immunogenic composition of the second aspect, for use in preventing, treating or ameliorating an infection, disease, disorder or condition.

In a seventh aspect of the present disclosure, there is provided a use of a non-lamellar lyotropic liquid crystalline phase carrier of the first aspect, or the immunogenic composition of the second aspect, in the manufacture of a medicament for the prevention, treatment or amelioration of an infection, disease, disorder or condition.

The various features and embodiments of the present disclosure, referred to in individual sections above apply, as appropriate, to other sections, mutatis mutandis. Consequently, features specified in one section may be combined with features specified in other sections as appropriate.

Further features and advantages of the present disclosure will become apparent from the following detailed description.

### BRIEF DESCRIPTION OF DRAWINGS

In order that the invention may be readily understood and put into practical effect, preferred embodiments will now be described by way of example with reference to the accompanying figures wherein:

FIG. 1. (A) Lattice parameter of the monoolein (MO)-based cubosomes following addition of trehalose-6,6-dimycolate (TDM) (0.4-10 mol %) at 25° C. in PBS. Different phases are identified as follows: primitive bicontinuous cubic (Q_{II}^{P}) and diamond bicontinuous cubic (Q_{II}^{D}). (B) Representative 1D SAXS patterns of intensity vs q for MO cubosomes and MO-TDM cubosomes (0.4-10 mol %).

FIG. 2. Representative Cryo-TEM images and fast Fourier transformation of (A) MO-cubosomes, (B) MO-TDM (1 mol %) cubosomes. Total lipid concentrations of all cubosomes were 50 mg/mL. Scale bars=200 nm.

FIG. 3. Hela and THP-1 cell viability in presence of MO and MO-TDM (1 mol %) cubosomes at 20 μg/ml after 72 hours of incubation. Cell viability calculated as a percentage (%) of control. The percentage (%) cell viability data are presented as a mean t standard deviation (SD) of two independent experiments with duplicate analyses of each sample.

FIG. 4. Solid state X-Ray diffraction patterns of MO, TDM and MO-TDM (1 mol %), collected from drop cast films on glass coverslips.

FIG. 5. Differential scanning calorimetric (DSC) scan analysis of MO, TDM and MO-TDM (1 mol %) mixture

FIG. 6. FTIR spectral (700-4000 cm⁻¹) analysis of MO, TDM and mixture of MO-TDM (1 mol %)

FIG. 7. (a) proinflammatory cytokines Interleukin (IL)-6 and tumor necrosis factor alpha (TNF-α) were measured after MTB infection and concurrently stimulated with MO, TDM and MO-TDM (1 mol %). (b) Macrophages were incubated with MO, TDM and MO-TDM (1 mol %) for three days before MTB infection. After 24 hours of infection proinflammatory cytokines IL-6 and TNF-α were measured. (c and d) MTB burden in macrophages pre or concurrently stimulated with MO, TDM and MO-TDM (1 mol %) as measured through colony-forming unit (CFU) assay over a period of 7 days of infection. Data represent the average of three independent experiments carried out in duplicate. Bars and error bars represent means and SD, respectively. Statistical analysis was performed with Paired two-tailed Student's t-test/one-way ANOVA with post-hoc analysis *p<0.05, **p<0.005, ***p<0.0005, ****p<0.0001. n.s. represent non-significance.

FIG. 8. Monocytes were trained with culture medium only (as a negative control), or with MO, TDM or MO-TDM (1 mol %) for 18 hours and then rested for five days. On the sixth day after re-stimulation with BCG, (a) the concentration of IL-6 and TNF-α cytokines were measured and, (b) the enrichment of H3K4me3 on the IL-6 and TNF-α promoter were measured by using chromatin immunoprecipitation (ChIP)—quantitative polymerase chain reaction (qPCR). Data represents the average of three independent experiments carried out in duplicates. Bars and error bars represent means and SD, respectively. Statistical analysis was performed with Paired two-tailed Student's t-test/one-way ANOVA with post-hoc analysis *p<0.05, **p<0.005, ***p<0.0005, ****p<0.0001. n.s. represent non-significance.

FIG. 9. Secreted levels of IL-2 by M. tuberculosis Ag85B specific T cells during co-culture with MO, TDM, MO-TDM (1 mol %) and untreated macrophages at different time post infection representing their antigen presentation capacity. During concurrent treatment (a) macrophages were incubated with MO, TDM and MO-TDM (1 mol %) at the beginning of infection whereas during pre-treatment (b) they were incubated with MO, TDM and MO-TDM for three days before MTB infection. Data shown are of macrophages from a single donor but are representative of three separate donors. Bars and error bars represent means and SD, respectively. Statistical analysis was performed with Paired two-tailed Student's t-test/one-way ANOVA with post-hoc analysis. *p<0.05, **p<0.005, ***p<0.0005, ****p<0.0001. n.s. represent non-significance.

FIG. 10A. Mathematical model description and calibration. (a) Schematic shows the key system interactions and variables of the model. The model contains two compartments: plasma and lymphatic, which communicate via antigen presenting cells (APCs) and antibodies. Antigen is injected into the plasma compartment intraperitoneally, and MTB is introduced as an I.V. bolus, and innate and adaptive response to the antigen and MTB is modelled. The antigen and MTB are not present simultaneously in the plasma compartment but are depicted so only to demonstrate all the possible interactions occurring in the model. In fact, MTB is introduced into the system only when all the antigen has been cleared, i.e., 60 days after antigen injection. (b) Calibration of the innate immune response (i.e., IL-6 and TNF-α induced neutralization of MTB) component of the model with in vitro data following exposure of MTB to macrophages, preincubated (Pre; top row) or concurrently (Con; bottom row) administered with (TDM, MO, or MO-TDM (1 mol %), or without (none) antigens. The inset in panels ii and iii (top row) shows the enlarged region demarcated by the dashed box to highlight the kinetics of cytokines during the incubation period (2 days). (c) Calibration of the antigen presentation process with in vitro data involving IL-2 secretion from effector CD4+ T-cells upon antigen presentation by unprimed (none) or primed (TDM, MO, or MO-TDM (1 mol %)) macrophages. The in vitro data (markers) used for the above calibrations is shown in FIGS. 8 and 9.

FIG. 10B. Model-based predictions in vivo. In vitro-in vivo extrapolation of the calibrated model showing innate and adaptive immune response in mice to intraperitoneally injected antigen on day 0, followed by rechallenge with MTB on day 60. Concentration kinetics of (a) antigen, (b) MTB, (c) IL-6, (d) TNF-α, and (h) antibodies in the plasma compartment is shown up to 120 days, with the insets highlighting the kinetics during the first 48-72 hours. Concentration kinetics of (e) effector CD4+ T-cells, (f) IL-2, and (g) plasma cells in the lymphatic compartment is shown up to 120 days.

FIG. 11. OVA IgG antibody response of mice twice immunised with cubosomes-OVA, modified cubosomes-OVA, PSNPs-OVA 50 nm or PSNPs-OVA 500 nm. Serum was harvested from final bleeds of twice immunised mice and antigen specific OVA IgG antibodies were assessed using ELISA assay. Serum was serially diluted 1 in 2 starting at 1 in 200 dilution. Data shown are of the antibody responses measured as optical density readings for each dilution and an average per group calculated.

### DESCRIPTION OF EMBODIMENTS

The present disclosure is predicated, at least in part, on the realisation that non-lamellar lyotropic liquid crystalline phase carriers (‘carriers’) may be particularly suitable for the delivery of antigens as part of a subunit vaccine composition and are surprisingly potent in terms of the generation of an immune response based on efficient delivery of the antigen of interest. One or more of the chemical and/or physical properties and/or architecture of the non-lamellar lyotropic liquid crystalline phase carrier, such as the nature of the component lipids, internal or average spontaneous curvature, charge, loading efficiency and micro-rheology, may advantageously provide a carrier which is capable of one or more of: (i) improved encapsulation and/or solubility of antigen; (ii) protection of antigen from damage or binding which would otherwise occur and inactivate or reduce the activity of said antigen; (iii) reduction in toxicity of the antigen compared with administration of the free antigen; and (iv) improvement in the observed immunogenicity of the delivered antigen compared with delivery of the free antigen. The formation of non-lamellar lyotropic liquid crystalline phase carriers which can be tailored to improve delivery of specific antigens will allow for widespread use within medical applications including immunisations.

In certain examples, the antigen may be suitable for generating an immune response, such as an adaptive immune response, against TB infection. Mycobacterial components that could be used in new tuberculosis vaccines remain largely unknown. An underexplored tuberculosis vaccine candidate is mycolic acid, or cord factor trehalose 6,6′ dimycolate (TDM), a lipid component abundant in the TB cell wall that is known to strongly stimulate host inflammatory responses, and granuloma formation. Although TDM is one of the oldest and best studied virulence factors of TB, its high toxicity and low aqueous solubility have severely limited its development as a possible subunit vaccine.

It is shown herein that non-lamellar lyotropic liquid crystalline phase carriers can be designed which are capable of delivering such an antigen to a host. The design of the carriers herein may, particularly though not exclusively, lend themselves to the delivery of an antigen in active form to facilitate generation of an immune response. Further, toxicity of an antigen may be reduced when administered to a subject and not only is an innate immune response observed, which may be initiated by the carrier itself, but also an adaptive response is observed indicating successful delivery to cells.

Induction of the desired immune response furthermore requires antigen delivery to professional antigen-presenting cells and activation of these cells. Delivery systems, such as carriers, and immune potentiators together determine the magnitude and quality of the innate immune response and the uptake and processing of the antigens by antigen-presenting cells. The non-lamellar lyotropic liquid crystalline phase carriers disclosed herein are shown to have high surface-to-volume ratio which provides several advantages including, increased bioavailability, dose proportionality, and reduced toxicity relative to the antigen alone. The structure of the non-lamellar lyotropic liquid crystalline phase carriers also enables antigens of different compositions and physical characteristics to be encapsulated.

Addition of a hydrophobic component to certain non-lamellar lyotropic liquid crystalline phase carriers, such as cubosomes, has been shown in the literature to negatively impact upon the internal nanostructure of the carrier via changes to the intrinsic curvature of the lipid bilayer (Strachan et al. Australian Journal of Chemistry 2020, 73, pages 1042-1050; Freire et al. Journal of Colloid and Interface Science 2021, 5%, pages 352-363). It would have been expected, in light of this knowledge, that large or long chain hydrophobic components would therefore produce a pronounced effect in such carriers. Surprisingly, the examples herein demonstrate that even a very long chain (C-80-90) fatty acid chain as is associated with TDM did not negatively impact on the carrier nanostructure and allowed for effective delivery.

Unless defined otherwise, all technical and scientific terms used herein have the same meaning as would be commonly understood by those of ordinary skill in the art to which this invention belongs.

In this patent specification, the terms ‘comprises’, ‘comprising’, ‘includes’, ‘including’, or similar terms are intended to mean a non-exclusive inclusion, such that a method or composition that comprises a list of elements does not include those elements solely, but may well include other elements not listed.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “non-lamellar lyotropic liquid crystalline phase carrier”, as used herein, refers to a self-assembled nonlamellar liquid crystalline phase, formed from at least one amphiphile to give a two and/or three-dimensional mesophase structure which is capable of carrying an antigen. Non-lamellar lyotropic liquid crystalline phase carriers are shown herein to promote an immune response through delivery of an antigen in an active form to the host system. The terms “lipid carrier”, “non-lamellar lyotropic liquid crystalline phase carrier”, “non-lamellar LLC carrier”, “lyotropic liquid crystalline (LLC) lipid carrier”, and “carrier” are used interchangeably herein. Such carriers do not, as is understood in the art, include liposomes. The term nonlamellar refers to the lyotropic liquid crystalline phase or lipid carrier or particle not being a liposome (or L phase) i.e. not presenting a planar lipid bilayer structure as is the case with a ‘classic’ liposome structure. Liquid crystalline phases, as described herein, are substances that exhibit a phase of matter that has properties between those of a conventional liquid, and those of a solid crystal. There are different types of liquid crystalline phases, which can be distinguished based on their different optical properties and other properties as are known in the art.

In embodiments, the non-lamellar lyotropic liquid crystalline phase carriers of the invention comprise only liquid crystals. That is, the non-lamellar lyotropic liquid crystalline phase carriers of the invention do not comprise any solid lipid component. The non-lamellar lyotropic liquid crystalline phase carriers of the invention are therefore not solid lipid nanocarriers (SLNs).

In embodiments, the term “non-lamellar lyotropic liquid crystalline phase carriers” may be used to encompass only cubic, hexagonal and sponge morphologies. While the “sponge phase” or “sponge particles” (L₃) are recognised as not possessing long range order and demonstrating equivalent crystalline periodicity of the inverse bicontinuous cubic phase (Q_{II}), they are often considered as a “melted” Q_{II} cubic phase and so are considered to be included as particles of the first aspect. Therefore, short range order sponge phases are explicitly considered to be within the scope of this term. In embodiments, the term “non-lamellar lyotropic liquid crystalline phase carriers” may be used to include one or more phases selected from the group consisting of hexagonal (normal and reversed), cubic (normal discrete, reversed discrete, reversed bicontinuous—including primitive, gyroid and diamond—and reversed discontinuous), and other ‘intermediate phases’ including the ribbon, mesh, or non-cubic ‘sponge’ bicontinuous phases.

The terms “amphiphile”, “amphiphilic” and “amphiphilic lipid”, as used herein refer to compounds which comprise both a hydrophilic and a hydrophobic moiety and may be employed as lipids, in formation of the non-lamellar lyotropic liquid crystalline phase carriers described herein. Typically, such compounds will have a hydrophilic head group and a hydrophobic tail. Suitable examples include fatty acids and a range of lipid molecules.

The term “pharmaceutically acceptable salt”, as used herein, refers to salts of the one or more active agents which are toxicologically safe for systemic or localised administration such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

According to a first aspect of the invention, there is provided a non-lamellar lyotropic liquid crystalline phase carrier comprising one or more lipids forming the carrier and an antigen associated with the carrier.

### Carrier

In embodiments, the non-lamellar lyotropic liquid crystalline phase carrier of the first aspect is formed by the self-assembly of the one or more lipids which, in embodiments, may be amphiphilic lipids. It will be understood that appropriate amphiphilic lipids will self-assemble when in the presence of an aqueous solution, such as water or an aqueous buffer solution, to form a lyotropic liquid crystalline structure displaying a non-lamellar mesophase.

In embodiments, the non-lamellar lyotropic liquid crystalline phase carrier of the first aspect comprises at least one amphiphilic lipid, at least two amphiphilic lipids, at least three amphiphilic lipids, at least four amphiphilic lipids, at least five amphiphilic lipids, at least six amphiphilic lipids, or at least seven amphiphilic lipids.

In embodiments, the non-lamellar lyotropic liquid crystalline phase carrier of the first aspect may consist of, or consist essentially of, one, two, three, four or five amphiphilic lipids. In this context, the expression “consisting essentially of” will be understood to mean the lyotropic liquid crystalline phase carrier consists of one, two, three, four or five amphiphilic lipids forming the carrier, and does not consist of any other lipids that form the carrier.

In embodiments, the non-lamellar lyotropic liquid crystalline phase carrier is formed by the self-assembly of the one or more amphiphilic lipids in the presence of the antigen. It will be appreciated that there may be multiple ways in which the antigen can be associated with, such as attached to, incorporated or encapsulated within, the carrier and the final approach will depend on the nature of the antigen and the manner in which the carrier is to deliver it. For example, in certain embodiments, it may be appropriate to focus on attachment of the antigen to largely the surface of the particle. Typically, however, the carrier will be formed in the presence of the antigen so that the antigen is incorporated within the lipid bilayer or the internal channels and folds of the carrier in addition to any incidental surface-bound antigen.

In embodiments, the non-lamellar lyotropic liquid crystalline phase carrier may be a colloidal carrier, being one with a particle size of less than 10 micrometers.

In embodiments, the particle size of the carrier of the first aspect may be between about 10 micrometers and about 40 nanometers. Preferably, the particle size is between about 5 micrometers and about 50 nanometers, more preferably between about 1 micrometer and about 50 nanometers, even more preferably between about 800 nanometers and about 50 nanometers, still more preferably between about 600 nanometers and about 50 nanometers, even yet more preferably between about 500 nanometers and about 50 nanometers or between about 400 nanometers and about 50 nanometers, or between about 5 micrometers and about 80 nanometers, more preferably between about 1 micrometer and about 80 nanometers, even more preferably between about 800 nanometers and about 80 nanometers, still more preferably between about 600 nanometers and about 80 nanometers, even yet more preferably between about 500 nanometers and about 80 nanometers or between about 400 nanometers and about 80 nanometers, or between about 5 micrometers and about 100 nanometers, more preferably between about 1 micrometer and about 100 nanometers, even more preferably between about 800 nanometers and about 100 nanometers, still more preferably between about 600 nanometers and about 100 nanometers, even yet more preferably between about 500 nanometers and about 100 nanometers or between about 400 nanometers and about 100 nanometers. The particles of the first aspect may therefore operate as nanocarriers of the one or more active agents within embodiments of the above particle size ranges.

In embodiments, the non-lamellar lyotropic liquid crystalline phase carrier has a bulk phase selected from the group consisting of the cubic phase, the hexagonal phase and the sponge phase, including normal and inverse/reverse phases of each, as appropriate.

These carrier matrices offer a range of advantages compared to their lamellar analogues, such as liposomes, in the delivery of antigens. Their lipid composition can render them more fusogenic with the outer membrane of appropriate cells and, owing to their high internal surface area and amphiphilic nature, non-lamellar lyotropic liquid crystalline phase carriers such as cubosomes have the capacity to encapsulate and release an array of antigens. Such carrier matrices can also protect the structural integrity of the encapsulated antigen from enzymatic degradation and can reduce the antigen's innate toxicity allowing for appropriate use as a subunit vaccine.

In embodiments, the non-lamellar lyotropic liquid crystalline phase particle is one selected from the group consisting of hexagonal (normal and reversed), cubic (normal discrete, reversed discrete, reversed bicontinuous—including primitive, gyroid and diamond—and reversed discontinuous), and other ‘intermediate phases’ including the ribbon, mesh, or non-cubic ‘sponge’ bicontinuous phases.

In embodiments, the non-lamellar lyotropic liquid crystalline phase carrier of the first aspect may be a cubosome or a hexosome.

In embodiments, the non-lamellar lyotropic liquid crystalline phase carrier is a cubosome.

In embodiments, the non-lamellar lyotropic liquid crystalline phase carrier is a hexosome.

Preferably, the cubosome is a bicontinuous cubic phase (V₁) or inverse bicontinuous cubic phase (V₂) cubosome. Inverse bicontinuous cubic phase (V₂) cubosomes are particularly preferred. It will be appreciated by a person skilled in the art that V₂ is an umbrella term for the varying cubic phases. V₂ can also be referred as Vu or Q_{II}. Within Q_{II} there are Q_{II}^{D}(Pn3m), Q_{II}^{P} (Im3m), Q_{II}^{G}(Ia3d).

Inverse (reverse) phase carriers may be preferred as they provide for a complex series of internal channels which can accommodate one or more active agents and which allow for a better controlled release profile in certain circumstances.

The cubic phase structure within cubosomes provides a lipid bilayer motif repeatedly wrapped to a triply periodic minimal surface. The increased surface curvature of the lipid membrane within these carriers of the first aspect may assist in promoting bilayer fusion upon contact with other self-assembled systems, including lipid membranes. High curvatures values are therefore preferred in the carriers of the present disclosure. Owing to their high internal surface area and amphiphilic nature, cubosomes have the capacity to encapsulate and release a wide array of antigens.

In terms of the lipids used in formation of the carrier of the first aspect, the critical packing parameter (CPP) of the lipid(s) can be used to rationalise the mean and Gaussian curvatures, being a property of the formed particle, and so indicate the nature of the mesophase formed or being formed and allows for considerations of suitability of the resulting non-lamellar lyotropic liquid crystalline phase carrier as an antigen carrier. The CPP is related to the mean and Gaussian curvatures via the following equation:

$$CPP = 1 + {Hl}_{c} + \frac{{Kl}_{c}^{2}}{3}$$

where:
lc is the effective length of the hydrocarbon chain
H is the Mean curvature
K is the Gaussian curvature
The molecular geometry of the relevant amphiphile lipid for use in forming the particles of the disclosure should satisfy the above equation to yield CPP >1.

The same approach can be applied for more than one amphiphile lipid. For multiple amphiphiles an amphiphile with an intrinsic CPP less than 1 can be included to a composition with a secondary amphiphile CPP greater than 1, such that the average CPP is greater than 1. Secondary additives which may also contribute to curvature increase to achieve CPP greater than 1 include small hydrophobic molecules and polymers which interact with the amphiphile headgroup. Conversely, the curvature can be decreased through inclusion of amphiphiles with CPP less than 1, high molecular weight PEG, strong chaotropes, charged headgroups, and solvents with a LogP between −1.5 and 0.

The non-lamellar lyotropic liquid crystalline phase carriers may thereby be classified based upon their interfacial curvature which may be calculated by approaches known in the art. In general terms, the curvature of the inverse lyotropic phases increases in the order lamellar <bicontinuous cubic <hexagonal <micellar cubic.

In embodiments, the one or more amphiphilic lipids have a critical packing parameter (CPP) about or greater than 1.0.

In embodiments, the one or more amphiphilic lipids have a CPP of between about 1.0 to about 3.0, preferably between about 1.0 to about 2.5, more preferably between about 1.0 to about 2.0, even more preferably between about 1.0 to about 1.75, still yet more preferably between about 1.0 to about 1.5.

It will be appreciated that when multiple lipids are incorporated into a carrier of the first aspect then all references above to CPP values of individual lipids become a reference to the average CPP value. That is, when the LLC particle comprises more than one amphiphile (lipid), an average CPP may be defined as the molar average of all the CPP values of the constituent amphiphile lipids. The average CPP values may be selected from those provided above.

In embodiments, the non-lamellar lyotropic liquid crystalline phase carrier has an average CPP value between about 1.0 to about 3.0, preferably between about 1.0 to about 2.5, more preferably between about 1.0 to about 2.0, even more preferably between about 1.0 to about 1.75, still yet more preferably between about 1.0 to about 1.5.

The CPP is calculated as follows: v/a₀l_{c}; where l_{c} is the effective length of the amphiphile (lipid) chain; a₀ is the effective surfactant head group area (determined by the balance of inter-chain attractive and head group repulsive interactions); and v is the average volume occupied by the amphiphile molecule.

Similarly, the spontaneous splay value correspondences to the spontaneous curvature of the non-lamellar LLC particle. When two lipids have corresponding spontaneous splay energies then fusion between them becomes more energetically favourable. It is therefore believed that particles of the first aspect having the following splay values will be more likely to undergo a desired fusion event with a biological membrane.

The choice of amphiphilic lipid will clearly affect splay and can be determined based on, for example, the selection of hydrophobes to enhance chain splay including employing unsaturated hydrophobes such as myristyl, pentadecenyl, oleyl, elaidyl, linoleyl, linolenyl, arachindonyl, docosenyl and/or isoprenoid-type hydrophobes such as 3,7,11-trimethyl-dodecyl, 5,9,13-trimethyltetradecanyl, 3,7,11,15-tetramethyl-hexadecyl, 5,9,13,17-tetramethyloctadecyl. Non-limiting examples of such lipids include ME, MP, MM, MV, MO, ML and MR, as are known in the art.

The energy cost of per surface area due to splay can be approximated by

$${f}_{s} = \frac{1}{2} \cdot κ \cdot {( div n )}^{2}$$

Where κ is the splay modulus of the monolayer [symbol] is the spontaneous splay.

$${f}_{s} = \frac{1}{2} \cdot {κ}_{t} \cdot {t}^{2}$$

κₜ is the splay modulus of the monolayer, t is the tilt vector.
The total energy cost

$${f}_{tot} = \frac{1}{2} \cdot κ \cdot {( div n )}^{2} + \frac{1}{2} \cdot {κ}_{t} \cdot {t}^{2} - \frac{1}{2} \cdot κ \cdot {2}^{}$$

In embodiments, the non-lamellar lyotropic liquid crystalline phase carrier has an internal curvature induced splay ( [symbol] ) less than −0.05 nm⁻¹.

In embodiments, the non-lamellar lyotropic liquid crystalline phase carrier has an internal curvature induced splay ( [symbol] ) less than −0.10 nm⁻¹.

In embodiments, the non-lamellar lyotropic liquid crystalline phase carrier has an internal curvature induced splay ( [symbol] ) less than −0.15 nm⁻¹.

In embodiments, the non-lamellar lyotropic liquid crystalline phase carrier has an internal curvature induced splay ( [symbol] ) less than −0.20 nm⁻¹.

In embodiments, the non-lamellar lyotropic liquid crystalline phase carrier has an internal curvature induced splay ( [symbol] ) less than −0.25 nm⁻¹.

In embodiments, the non-lamellar lyotropic liquid crystalline phase carrier has an internal curvature induced splay ( [symbol] ) between about −0.05 nm⁻¹ to about −0.95 nm⁻¹, or between about −0.05 nm⁻¹ to about −0.85 nm⁻¹, or between about −0.05 nm⁻¹ to about −0.75 nm⁻¹, or between about −0.05 nm⁻¹ to about −0.65 nm⁻¹, or between about −0.05 nm⁻¹ to about −0.55 nm⁻¹, or between about −0.05 nm⁻¹ to about −0.40 nm⁻¹, or between about −0.10 nm⁻¹ to about −0.95 nm⁻¹, or between about −0.10 nm⁻¹ to about −0.85 nm⁻¹, or between about −0.10 nm⁻¹ to about −0.75 nm⁻¹, or between about −0.10 nm⁻¹ to about −0.65 nm⁻¹, or between about −0.10 nm⁻¹ to about −0.55 nm⁻¹, or between about −0.10 nm⁻¹ to about −0.40 nm⁻¹, or between about −0.15 nm⁻¹ to about −0.75 nm⁻¹, or between about −0.15 nm⁻¹ to about −0.65 nm⁻¹, or between about −0.15 nm⁻¹ to about −0.55 nm⁻¹, or between about −0.15 nm⁻¹ to about −0.40 nm⁻¹, or between about −0.20 nm⁻¹ to about −0.75 nm⁻¹, or between about −0.20 nm⁻¹ to about −0.65 nm⁻¹, or between about −0.20 nm⁻¹ to about −0.55 nm⁻¹, or between about −0.25 nm⁻¹ to about −0.75 nm⁻¹, or between about −0.25 nm⁻¹ to about −0.65 nm⁻¹, or between about −0.25 nm⁻¹ to about −0.55 nm⁻¹.

Further, a person of skill in the art will, in light of the present disclosure, be able to use the following equation to ascertain appropriate curvature levels to provide the benefits described herein:

$${c}_{0 T} = {xc}_{0 i} + ( 1 - x ) {c}_{0 j}$$

where c_{0T} is total curvature, x is fractional composition lipid i, c₀ᵢ is curvature of lipid i, c₀ⱼ, is curvature of lipid j, (1-x) is fractional composition of lipid j. For further combinations the equation is expanded to include lipid k; where the sum of compositions equals 1.

In embodiments, the lattice parameter of the non-lamellar lyotropic liquid crystalline phase carrier is between about 20 to about 684 Å, or between about 20 to about 500 Å, or between about 20 to about 400 Å, or between about 20 to about 200 Å, or between about 20 to about 190 Å, or between about 20 to about 180 Å, or between about 20 to about 170 Å, or between about 20 to about 160 Å, or between about 20 to about 150 Å, or between about 40 to about 684 Å, or between about 40 to about 500 Å, or between about 40 to about 400 Å, or between about 40 to about 200 Å, or between about 40 to about 190 Å, or between about 40 to about 180 Å, or between about 40 to about 170 Å, or between about 40 to about 160 Å, or between about 40 to about 150 Å, or between about 60 to about 684 Å, or between about 60 to about 500 Å, or between about 60 to about 400 Å, or between about 60 to about 200 Å, or between about 60 to about 190 Å, or between about 60 to about 180 Å, or between about 60 to about 170 Å, or between about 60 to about 160 Å, or between about 60 to about 150 Å, or between about 80 to about 684 Å, or between about 80 to about 500 Å, or between about 80 to about 400 Å, or between about 80 to about 200 Å, or between about 80 to about 190 Å, or between about 80 to about 180 Å, or between about 80 to about 170 Å, or between about 80 to about 160 Å, or between about 80 to about 150 Å, or between about 100 to about 684 Å, or between about 100 to about 500 Å, or between about 100 to about 400 Å, or between about 100 to about 200 Å, or between about 100 to about 190 Å, or between about 100 to about 180 Å, or between about 100 to about 170 Å, or between about 100 to about 160 Å, or between about 100 to about 150 Å, or between about 120 to about 684 Å, or between about 120 to about 500 Å, or between about 120 to about 400 Å, or between about 120 to about 200 Å, or between about 120 to about 190 Å, or between about 120 to about 180 Å, or between about 120 to about 170 Å, or between about 120 to about 160 Å, or between about 120 to about 150 Å.

Lattice parameters can, for example in relation to sponge particles which typically have larger lattice parameters than cubosomes or hexosomes, be swollen in ranges up to 684 Å. More commonly swollen phases may have values from 200-400 Å. Swollen lattice parameters have certain design rules including that the head group could contain electrostatic charges and these can be negatively charged (e.g. PG and PS phospholipids) or positively charged (e. g. DOTAP (1,2-dioleoyl-3-trimethylammonium propane) and DODMAC (dimethyldioctadecylammonium chloride)). The head group may comprise hydration agents with multiple hydroxyl groups (e.g. DGMO and OG). The hydrophobic region may comprise cholesterol or other stiffening agents to stabilise the membrane and/or may comprise amphiphiles that promote a decrease in membrane curvature (e.g. PC and PE phospholipids). Lipid-PEG polymers (e.g. DOPE-PEG and MO-PEG) could be used in combination with charged lipids to swell the water channels. Further, block copolymers (e.g. Pluronic F127, F108 and Polysorbate 80) could be used as stabilisers when nanoparticle dispersions are required, although they may not have a direct effect on swelling the water channels.

The one or more amphiphilic lipids forming the carrier of the first aspect may be selected from those which are known in the art to form, particularly, cubosomes and hexosomes. The selection of the appropriate one or more amphiphilic lipids may be made on the basis of certain requirements which are understood in the art. For example, the lipid(s) may be chosen from those which adopt a Type II lyotropic liquid crystalline phase at ambient and physiological temperatures. Parameters which may be appropriate for selection of an appropriate lipid include (i) on the hydrophobic component: 1. The temperature should be above the chain melting temperature such that molten chains are present; and 2. There should be at least one cis unsaturated bond in a carbon chain of at least 14 carbons at a position at least mid-way along the backbone; or 3. The carbon backbone should contain at least 12 carbons of which 3 are secondary carbons with methyl branches; and 4. The molecular weight of the hydrophobe should be at least greater than 200 amu; and (ii) in relation to the head group: 5. The head group should contain at least three functional groups with minimum hydrophilicity (e.g. hydroxyl); 6. The head group should be able to form head group-water hydrogen bond networks; and 7. The head group area should be small relative to the hydrophobe footprint. By way of a guide, this is exemplified by the MO lipid used in the examples of the present disclosure as it: fulfils criteria 1, 2 and 4 for the hydrophobe; and fulfils criteria 5, 6 and 7 for the head group. It will be appreciated that many other lipids are available which fulfil these criteria appropriately and they may be selected on the basis of these criteria which are known, or easily ascertained, values.

Guidance may be found in one or more of the following publications which are each incorporated by reference herein in their entirety: (i) T. Kaasgaard and C. J. Drummond “Ordered 2D and 3D Nanostructured Amphiphile Self-Assembly Materials Stable in Excess Solvent” Phys. Chem. Chem. Phys. 2006, 8, pages 4957-4975. (ii) C. Fong, T. Le and C. J. Drummond “Lyotropic Liquid Crystal Engineering—Ordered Nanostructured Small Molecule Amphiphile Self-Assembly Materials by Design” Chem. Soc. Rev., 2012, 41, pages 1297-1322; (iii) L. van ‘t Hag, S. L. Gras, C. E. Conn and C. J. Drummond “Lyotropic liquid crystal engineering moving beyond binary compositional space—Ordered nanostructured amphiphile self-assembly materials by design” Chem. Soc. Rev., 2017, 46, pages 2705-2731; and (iv) S. Sarkar, N. Tran, Md H. Rashid, T. C. Le, I. Yarovsky, C. E. Conn and C. J. Drummond “Toward cell membrane biomimetic lipidic cubic phases: a high-throughput exploration of lipid compositional space” ACS Applied Biomaterials, 2019, 2, pages 182-195.

Poly-hydroxyl (glycolipids) and polyethers (polyethylene oxides) form two of the largest categories of Type II forming head groups. Non-limiting examples of head group motifs include alcohols, fatty acids, monoacylglycerides, MAGs, 2-MAGs, glycerates, glyceryl ethers, ethylene oxides, amides, monoethanolamides, diethanolamides, serinolamides, methylpropanediolamides, ethylpropanediolamides, ureas, urea alcohols, biurets, biuret alcohols, ureides, endocannabinoids (anandamide, virodhamine, 2-glycerol, dopamine, 2-glycerol ether) and glycolipids. Examples include phospholipids such as DMPC and DMPE.

In embodiments, the one or more amphiphilic lipids may be selected from the group consisting of ethylene oxide-, monoacylglycerol-, glycolipid-, phosphatidylethanolamine-, and urea-based amphiphiles, and derivatives or analogues thereof.

Ethylene oxide amphiphiles may include C₁₂(EO)₂, C₁₂(EO)₄, C₁₂(EO)₅, and C₁₂(EO)₆ and dialkyl ethylene oxide amphiphiles. Monoacylglycerols may include monomyristolein, monoolein, monovaccenin and monoerucin. Amphiphiles resembling monoacylglycerols may be appropriate and include oleyl glycerate, phytanyl glycerate, glyceryl monooleyl ether, glyceryl phytanyl ether, phytantriol and monononadecenoin. Glycolipids with sugar moieties which may be appropriate including monosubstituted glycolipids: β-Mal₃(Phyt)₂, β-Glc(Phyt), β-Xyl(Phyt), β-Glc-(TMO)₂, β-Mal₂(Phyt)₂ and β-Glc(Phyt)₂; and disubstituted unbranched glycolipids: 1,2-diacyl-(β-D-glucopyranosyl)-sn-glycerols; 1,2-dialkyl-(β-D-glucopyranosyl)-sn-glycerols; 1,3-diacyl-(β-D-glucopyranosyl)-sn-glycerols; 1,3-dialkyl-(β-D-glucopyranosyl)-sn-glycerols. Phosphatidylethanolamine amphiphiles may include dioleoylphosphatidylcholine (DOPC) and dioleoylphosphatidylethanolamine (DOPE). Urea amphiphiles may include dodecylurea (DU), octadecylurea (ODU), oleylurea (OU), oleylbiuret (OBU), linoleylurea (LU), phytanylurea (PU), hexahydrofarnesyl-urea (HFU).

In embodiments, the one or more amphiphilic lipids may be selected from the group consisting of 1-monoolein, 2-monoolein, citrem, oleoyl lactate, oleamide, monoelaidin, linoleic acid, elaidic acid, monopalmitolein, monolinolein, phytantriol, diolein, triolein, dioleoyl-glycerol, 1,2-Dioleoyl-3-trimethylammonium propane (DOTAP), N—N-dioleoyl-N, N-dimethylammonium chloride (DODAC), dioctadecyl ammonium chloride (DOAC), dioctadecyl dimethyl ammonium chloride (DODMAC) or dioctadecyl dimethyl ammonium bromide (DODAB), 1,2-dioleoyl-sn-glycero-3-phosphoethanolamine (DOPE), 1,2-Dioleoyl-phosphatidylglycerol (DOPG), oleic acid (OA), lysol-hydroxy-2-oleoyl-sn-glycero-3-phosphocholine (PC), 1,2-dioleoyl-sn-glycero-3-dihexyl-phosphocholine (DOPC), vitamin E tocopherol, vitamin E (tocopheryl) acetate, phytanoyl monoethanolamide, farnesoyl monoethanolamide, oleoyl monoethanolamide, linoleoyl monoethanolamide and linolenoyl monoethanolamide.

Single-chain amphiphile lipids may be selected from the group consisting of saturated fatty acids C7-C16, oleic acid, elaidic acid, linoleic acid, sodium/gadolinium oleate, oleamide, 1-glyceryl monooleyl ether (GME), GMO, 2-MO, oleoyl lactate, citrem, Diglycerol monooleate (DGMO), Lyso (1-oleoyl)-phosphatidyl-choline, (Z)-Octadec-9-enylferrocene, N-Dodecyl-caprolactam (C12), Vitamin Ki, ubiquinone-10 (coenzyme Q10), Vitamin E, Vitamin E acetate, Vitamin A palmitate, Alpha-tocopheryl PEO1000 succinate (vitamin E TPGS), PEG2000-MO, PEG-PT, PEOₓ-stearate (x=40-100), polysorbate 80.

Amphiphile lipids with multiple alkyl chains may be selected from the group consisting of didodecyldimethylammonium bromide (DDAB); Di(canola ethyl ester) dimethyl ammonium chloride (DEEDAC); Dioctadecyl (dimethyl) ammonium chloride (DODMAC), dioctadecyl ammonium chloride (DOAC) or dioctadecyl dimethyl ammonium bromide (DODAB); diolein; Dioleoyl-glycerol (DOG), EDTA-bi-oleoyl; EDTA-bi-phytanyl; 1,2-Dioleoyl-3-trimethylammonium-propane (DOTAP); 1,2-Dioleoyl-phosphatidic acid (DOPA); 1,2-Dioleoyl-phosphatidylglycerol (DOPG), 1,2-Distearoyl-phophatidylglycerol (DSPG); 1,2-Dioleoyl-phosphatidylethanolamine (DOPE), 1,2-distearoyl-glycero-3-phosphoethanolamine (DSPE); 1,2-Dioleoyl-phosphatidylcholine (DOPC); 1-Palmitoyl-2-oleoyl-sn-glycero-3-phosphocholine (POPC); 1,2-Dioleoyl-sn-glycero-3-phosphoserine (DOPS); 1,2-Dipalmitoylphosphatidylserine (DPPS); DSPE-mPEG350, 750, 2000 (X=7, 16 or 45); DSPE-PEG2000, 3400, 5000; DMPE-mPEG550; (C18)2 DTPA (Gd), Cardiolipin, Cyclodextrin derivative (βCD-nC₁₀).

In any embodiment herein, the amphiphilic lipid may be a monoolein and/or phytantriol.

In any embodiment herein, the carriers of the present disclosure may comprise MO or phytantriol in combination with one or more of cholesterol, DLPC, DSPC, DPPE, DPPS, DOPS, DPPC, DMPC, DMPS and DLPS.

Monoacylglycerols are known to form reversed phases over large regions of their phase diagrams, with monoolein being the most prominent. Formation of reversed phases is favoured because of the kink that is introduced by the cis-double bond. The longer acyl chain increases the hydrophobic chain volume and makes monoolein more wedge-shaped and shifted towards type 2 phases in the spectrum of mesophases. If the double bond is closer to the end of the lipid it diminishes its effect and makes it less wedge-shaped. Acyl chain extension is expected to drive the mesophase formation further towards the type 2 phases, and on this basis it is not surprising that the H2-phase becomes the dominant phase with such a change.

In embodiments, the lipids forming the non-lamellar lyotropic liquid crystalline phase carrier of the first aspect may substantially comprise monolein.

In embodiments, the lipids forming the non-lamellar lyotropic liquid crystalline phase carrier of the first aspect may substantially comprise monolein and/or phytantriol.

In embodiments, the lipids forming the non-lamellar lyotropic liquid crystalline phase carrier of the first aspect may substantially comprise monolein and/or phytantriol, and DOPE.

In embodiments, the lipids forming the non-lamellar lyotropic liquid crystalline phase carrier of the first aspect may substantially comprise monolein and/or phytantriol, and DOTAP.

In embodiments, the lipids forming the non-lamellar lyotropic liquid crystalline phase carrier of the first aspect may substantially comprise monolein and/or phytantriol, and TOAB.

In embodiments, the lipids forming the non-lamellar lyotropic liquid crystalline phase carrier of the first aspect may substantially comprise monolein and/or phytantriol, and oleic acid.

In embodiments, the lipids forming the non-lamellar lyotropic liquid crystalline phase carrier of the first aspect may substantially comprise monolein and/or phytantriol, and DOPE and DOTAP.

In embodiments wherein the non-lamellar lyotropic liquid crystalline phase carrier of the first aspect comprises DOPE then it may be present at between 10 to 40 mol %.

In embodiments wherein the non-lamellar lyotropic liquid crystalline phase carrier of the first aspect comprises DOTAP then it may be present at between 0.5 to 5 mol %, or 0.5 to 4 mol %.

In embodiments, the non-lamellar lyotropic liquid crystalline phase carrier of the first aspect comprises monoolein (80-99.9 mol %) and triolein (0.1-20 mol %).

In embodiments, the non-lamellar lyotropic liquid crystalline phase carrier of the first aspect comprises monoolein (80-99.9 mol %) and vitamin E (0.1-20 mol %).

In embodiments, the non-lamellar lyotropic liquid crystalline phase carrier of the first aspect comprises monoolein (80-99.9 mol %) and DOPE (0.1-20 mol %).

In embodiments, the non-lamellar lyotropic liquid crystalline phase carrier of the first aspect comprises monoolein (95-99.9 mol %) and DOTAP (0.1-5 mol %).

In embodiments, the non-lamellar lyotropic liquid crystalline phase carrier of the first aspect comprises monoolein (95-99.9 mol %) and DODAB (0.1-5 mol %).

### Stabilizer

In embodiments, the non-lamellar lyotropic liquid crystalline phase carrier may further comprise at least one stabiliser. The stabiliser is selected from those known in the art and may be useful in providing for steric stabilisation and/or reducing flocculation of the carriers.

In embodiments, the stabiliser is a poloxamer, or a modified version of these.

In embodiments, the stabiliser is a surfactant, or a modified version of these.

In embodiments, the stabiliser is a PEGylated lipid stabilizer, or a modified version of these. It will be apparent to the skilled person that reference to a PEGylated lipid is a lipid that has been modified with polyethylene glycol (PEG).

In embodiments, the stabiliser is selected from a PEG-PPO-PEG triblock copolymer and a non-ionic block copolymer surfactant and a PEO co-polymerised with a charged moiety. Poloxamer 407 and Pluronic 127 may be suitable examples of a stabilising agent and may be incorporated into any of the embodiments of the first aspect described herein. PEO co-polymerised with (3-Acrylamidopropyl)trimethylammonium chloride, or a similar charge-carrying moiety, may also be appropriate. PEGylated lipid stabilisers are also appropriate including but not limited to PEG2000-MO, PEG-PT, DSPE-PEG (2000) Amine, 18:0 PEG2000 PE, and DSPE-PEG (5000) Amine. Many other such stabilisers are known in the art.

Other PEGylated lipids which may be useful include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. In embodiments, a PEGylated lipid includes PEG-c-DOMG, PEG-DMG, PEG-DLPE, PEG-DMPE, PEG-DPPC, a PEG-DSPE lipid, and combinations thereof.

In embodiments, the nature of the stabiliser is selected based on the nature of the lipids, the selection and understanding of the compatibility of these components can be based upon information known in the art.

The many steric stabilisers which have been reported to date can be divided into four groups: (i) amphiphilic block copolymers (i.e. Poloxamer™), (ii) PEGylated lipids (iii) customized lipid-copolymers and (iv) alternative steric stabilizers (e.g., bile salts, proteins). Ideally the stabiliser selected will prevent aggregation of the particles by providing an electrostatic or, more commonly, steric barrier between approaching particles. Stabilisers which may function optimally in the lipid particles of the present disclosure share similar properties including (i) they are generally highly hydrophilic with a high HLB (hydrophilic-lipophilic balance) value due to an asymmetric amphiphilic polymer structure with a larger hydrophilic domain. It is important that the hydrophilic part of the molecule is not surrounded by hydrophobic regions. A high HLB may be achieved via use of longer PEG chains or multiple PEG chains; (ii) presence of hydrogen bond acceptors and absence of hydrogen bond donors; and (iii) electrically neutral. A person of skill in the art can select the appropriate stabiliser on this basis. Further, the following journal articles address key aspects of stabilisers which may be appropriate for use with the lipid particles of the present disclosure and are hereby incorporated by reference in their entirety: (i) J. Y. T. Chong, X. Mulet, B. J. Boyd and C. J. Drummond; “Steric Stabilizers for Cubic Phase Lyotropic Liquid Crystal Nanodispersions (Cubosomes)” in “Advances in Planar Lipid Bilayers and Liposomes”, Vol 21, Chp 5, (2015) p. 131-187, ISSN 1554-4516, Elsevier; (ii) J. Zhai, B. Fan, S. H. Thang, C. J. Drummond “Novel amphiphilic block copolymers for the formation of stimuli-responsive non-lamellar lipid nanoparticles” Molecules, 2021, 26, 3648-3664; (iii) J. Zhai, R. Suryadinata, B. Luan, N. Tran, T. M. Hinton, J. Ratcliffe, X. Hao and C. J. Drummond “Amphiphilic brush polymers produced by the RAFT polymerisation method stabilise and reduce the cell toxicity of lipid lyotropic liquid crystalline nanoparticles” Faraday Discussions, 2016, 191, 545-563; Faraday Discussion 191 on Nanoparticles with Morphological and Functional Anisotropy; (iv) J. Zhai, T. J. Hinton, L. J. Waddington, C. Fong, N. Tran, X. Mulet, C. J Drummond and B. W. Muir “Lipid-PEG Conjugates Sterically Stabilise and Reduce the Toxicity of Phytantriol-Based Lyotropic Liquid Crystalline Nanoparticles” Langmuir, 2015, 31, 10871-10880; (v) J. Y. T. Chong, X. Mulet, D. Keddie, L. J. Waddington, S. T. Mudie, B. J. Boyd and C. J. Drummond “Novel Steric Stabilisers for Lyotropic Liquid Crystalline Nanoparticles: Pegylated Phytanyl Copolymers” Langmuir, 2015, 31, 2615-2629; (vi) J. Y. T. Chong, X. Mulet, A. Postma, D. J. Keddie, L. J. Waddington, B. J. Boyd and C. J. Drummond “Novel RAFT Amphiphile Brush Copolymer Steric Stabilisers for Cubosomes: Poly(octadecyl acrylate)-block-poly(polyethylene glycol methyl ether acrylate)” Soft Matter, 2014, 10, 6666-6676; (vii) A. Tilley, C. J. Drummond and B. J. Boyd “Disposition and Association of the Steric Stabiliser Pluronic F127 in Lyotropic Liquid Crystalline Nanostructured Particle Dispersions” J. Colloid and Interface Science, 2013, 392, 288-296. (viii) J. Y. T. Chong, X. Mulet, L. J. Waddington, B. J. Boyd and C. J. Drummond “High Throughput Discovery of Novel Steric Stabilisers for Cubic Lyotropic Liquid Crystal Nanoparticle Dispersions” Langmuir, 2012, 28, 9223-9232; (ix) J. Y. T. Chong, X. Mulet, L. J. Waddington, B. J. Boyd and C. J. Drummond “Steric Stabilisation of Cubic Lyotropic Liquid Crystalline Nanoparticles: High Throughput Evaluation of Triblock Polyethylene Oxide-Polypropylene Oxide-Polyethylene Oxide Copolymers.” Soft Matter, 2011, 7, 4768-4777.

In embodiments, the stabiliser is present during formation of the carrier of the first aspect.

In embodiments, the stabiliser is present at between 1 to 20 wt % or 5 to 20 wt % of the carrier.

In embodiments, the stabiliser is present at between 6 to 18 wt %.

In embodiments, the stabiliser is present at between 7 to 16 wt %.

In embodiments, the stabiliser is present at between 8 to 14 wt %.

Cationic and/or Ionizable Lipids

In embodiments, the non-lamellar lyotropic liquid crystalline phase carrier may further comprise at least one additional cationic and/or ionizable lipids, for example one or more cationic and/or ionizable lipids comprising a cyclic or non-cyclic amine. The at least one additional cationic and/or ionizable lipid may be selected from those known in the art. The at least one additional cationic and/or ionizable lipid may be suitably selected such that they do not disrupt the structure of the non-lamellar lyotropic liquid crystalline phase carrier.

Ionizable lipids are a class of lipid molecules that remain neutral at physiological pH, but are protonated at low pH, making them positively charged. A wide range of ionizable lipids have been developed and are commercially available, as would be known to a person of skill in the art.

Without wishing to limit the scope of ionizable lipids suitable for use, typically such lipids will have an amino-containing head group which can be protonated at acidic pH values. A pKa value for the ionizable lipid may be between 5.5 to 7.2, preferably between 5.9 to 6.8. The ionizable lipid will typically also have at least one lipid chain but preferably there will be two or more such tails and branching in at least one tail has been demonstrated to provide desirable characteristics.

In embodiments, the ionizable lipid may be selected from those described in WO2017/218704, WO2018/078053, WO2015/199952, WO2018/081480, WO2017/117528, WO2018/081638, WO2018/107026, WO2019/089828, WO2020/081938, and WO2021/030701, which are hereby incorporated by reference in their entirety.

In certain embodiments of the disclosure, the ionizable lipid may be an amino lipid having the structure of Formula (I):

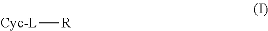

wherein:
Cyc is a nitrogen heterocycle or heteroaryl;
L is an amido-linker; and
R is a C₁₀ to C₄₄ carbon chain.

In embodiments, the amino lipid has the structure of Formula (Ib):

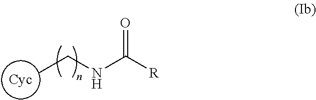

wherein:
Cyc is a 5- or 6-membered nitrogen heterocyclyl or heteroaryl, optionally selected from the group consisting of pyridyl, pyrimidinyl, piperidinyl, piperazinyl, morpholinyl, and pyrazinyl;
L is an amido-linker;
R is a C₁₀ to C₄₄ carbon chain, optionally a C₁₂ to C₂₄ alkyl or alkenyl, optionally interrupted by one or more heteroatoms; and
n is an integer from 1 and 6.

In embodiments, R is selected from the group consisting of oleyl, linoleoyl, linolenoyl, phytanoyl, and farnesoyl.

In embodiments, the amino lipid is selected from the group consisting of:

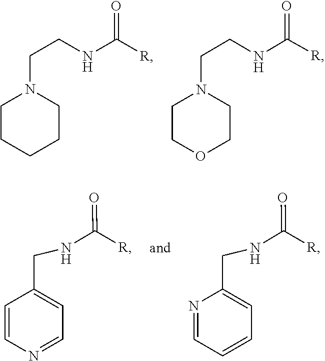

wherein, R is as defined above for Formula (I) or (Ib).

In certain embodiments, the cationic and/or ionizable lipid maybe selected from the non-limiting group consisting of: 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22). 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), (6Z,9Z,28Z,31Z)-heptatriacont-6,9,28,31-tetraene-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3p)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), (2S)-2-({8-[(3p)-cholest-5-en-3-yloxy]octyl}oxy)-N,N-dimethyl-3-[(9Z,12Z)-octadeca-9,12-die n-1-yloxy]propan-1-amine (Octyl-CLinDMA (2S)), ((4-hydroxybutyl)azanediyl)bis(hexane-6,1-diyl)bis(2-hexyldecanoate)) (ALC-0315), 8-[(2-hydroxyethyl)[6-oxo-6-(undecyloxy)hexyl]amino]-octanoic acid, 1-octylnonyl ester, 4-hydroxybutyl)azanediyl)bis(hexane-6,1-diyl)bis(2-hexyldecanoate)), heptadecan-9-yl 8-[2-hydroxyethyl-(6-oxo-6-undecoxyhexyl)amino]octanoate (SM-102), and dioleoyl-3-trimethylammonium propane (DOTAP).

In embodiments, the cationic and/or ionisable lipid is present during formation of the carrier of the first aspect.

The amount of cationic/ionisable lipids in the carrier may be suitably selected depending on, for example, the specific application, lipid composition, amounts of other carrier components such as helper lipids or polymers, and the desired properties of the carrier. Typically, the cationic and/or ionisable lipid may be present at between 0.5 to 10 wt % of the carrier.

In embodiments, the cationic lipid is present at between 0.5 to 5 wt % of the carrier. Advantageously, such amounts may be useful for maintaining a high stability of the carrier, when using highly efficient cationic and/or ionisable lipids that exhibit potent transfection or delivery capabilities, and/or for applications where a lower charge density or reduced toxicity is desired.

In embodiments, the cationic lipid is present at between 0.5 to 10 wt % of the carrier. Advantageously, such amounts may be useful for enhancing the interaction between the cationic and/or ionisable lipids and the antigen, which may lead to improved complex formation and cellular uptake.

### Antigen

In embodiments, the antigen is selected from the group consisting of a protein, a glycoprotein, a peptide, a glycopeptide, a polysaccharide, a lipid, a glycolipid, a lipoprotein, a lipopeptide, and a nucleic acid. In embodiments, the antigen is selected from the group consisting of a protein, a glycoprotein, a peptide, a glycopeptide, a polysaccharide, a lipid, a glycolipid, a lipoprotein, and a lipopeptide. In embodiments, the antigen is selected from the group consisting of a protein, a glycoprotein, a peptide, a polysaccharide, a lipid, and a glycolipid. In embodiments, the antigen is selected from the group consisting of an antigenic protein, peptide, glycoprotein or glycolipid.

When the antigen is an antigenic protein, peptide, glycoprotein or glycolipid then it may be derived from a pathogenic bacterial, viral or fungal organism or a cancer cell. Such organisms may include, but are not limited to, Streptococcus species, Candida species, Brucella species, Salmonella species, Shigella species, Pseudomonas species, Bordetella species, Clostridium species, Norwalk virus, Bacillus anthracis, Mycobacterium tuberculosis, human immunodeficiency virus (HIV), Chlamydia species, Human Papillomaviruses, Japanese encephalitis virus, Influenza virus, Paramyxovirus species, Herpes virus, Cytomegalovirus, Varicella-Zoster virus, Epstein-Barr virus, Hepatitis viruses, Plasmodium species, Trichomonas species, sexually transmitted disease agents, viral encephalitis agents, protozoan disease agents, fungal disease agents, prion disease agents and bacterial disease agents.

In embodiments, the antigen is hydrophobic or at least comprises at least one hydrophobic chain, for example one, two, three, four, five or six hydrophobic chains. In this context “hydrophobic” will be understood to mean the antigen has an overall hydrophobic character. Further, the terms “hydrophobic chain”, “aliphatic chain” and “lipophilic chain” may be used interchangeably herein. The hydrophobic chains may be capable of being associated with or embedded in a lipid bilayer.

Examples of suitable groups that can make up the hydrophobic chain include, but are not limited to, fatty acids (lipids) including mycolic acids, glycerolipids, glycerophospholipids, sphingolipids, steroids including sterols, terpenes and terpeniods, saccharolipids, polyketides, and poly(hydrophobic amino acids) (pHAAs). In embodiments, the antigen comprises one or more hydrophobic chains independently selected from fatty acids (lipids) including mycolic acids, glycerolipids, glycerophospholipids, sphingolipids, steroids including sterols, terpenes and terpeniods, saccharolipids, and polyketides. In embodiments, the antigen comprises one or more hydrophobic chains independently selected from fatty acids (lipids), glycerolipids, glycerophospholipids, and sphingolipids.

In embodiments, one or more of the hydrophobic chains comprise or are hydrocarbon chains, which may be saturated or unsaturated, and optionally oxygenated derivatives thereof. The hydrocarbon chain may be linear or branched, and may contain carbocyclic rings in the chain, for example cyclopropane. Each such hydrocarbon chain may independently contain at least 10, 20, 30, 40 or 50 carbon atoms in the chain. Each such minimum total carbon chain numbers may be combined with each of an upper limit of 100, 90, 80, 70, or 60 carbon atoms in the chain. In embodiments, one or more of the hydrophobic chains comprise or are hydrocarbon chains having from 10 to 100, from 10 to 90, from 10 to 80, from 10 to 70, from 10 to 60, from 20 to 100, from 20 to 90, from 20 to 80, from 20 to 70, from 20 to 60, from 30 to 100, from 30 to 90, from 30 to 80, from 30 to 70, from 30 to 60, from 40 to 100, from 40 to 90, from 40 to 80, from 40 to 70, from 40 to 60, from 50 to 100, from 50 to 90, from 50 to 80, from 50 to 70, or from 50 to 60 carbon atoms in the chain. Each hydrocarbon chain may be independently saturated or unsaturated. Each hydrocarbon chain may be independently linear or branched, and may include a carbocyclic ring such as a cyclopropane in the chain. Each hydrocarbon chain may independently include one, two, three or four oxygen interruptions in the chain. Each hydrocarbon chain may be independently substituted with one, two, three or four oxygen-containing functional groups, for example hydroxy, methoxy and ketone groups.

In embodiments, one or more of the hydrophobic chains comprise a carbocycle, which may be saturated or unsaturated, and include polycyclic carbocycles and include fused, bridged and spirocyclic systems. Examples of suitable carbocycles include, but are not limited to, monocyclic carbocycles, for example cyclopropane, and polycyclic carbocycles, for example steroids including sterols such as cholesterol. In embodiments, one or more of the hydrophobic chains comprise or consist of a carbocycle and a hydrocarbon chain.

In embodiments, the total number of carbon atoms in all such hydrophobic chains combined may be at least 20, 30, 40, 50, 60, 70, or 80 carbon atoms. Each such minimum total carbon chain numbers may be combined with each of an upper limit of 200, 150, or 100 carbon atoms. In embodiments, the total number of carbon atoms in the hydrophobic chains of the antigen combined is from about 20 to about 200 carbon atoms, from about 20 to about 150 carbon atoms, from about 20 to about 100 carbon atoms, from about 30 to about 200 carbon atoms, from about 30 to about 150 carbon atoms, from about 30 to about 100 carbon atoms, from about 40 to about 200 carbon atoms, from about 40 to about 150 carbon atoms, from about 40 to about 100 carbon atoms, from about 50 to about 200 carbon atoms, from about 50 to about 150 carbon atoms, from about 50 to about 100 carbon atoms, from about 60 to about 200 carbon atoms, from about 60 to about 150 carbon atoms, from about 60 to about 100 carbon atoms, from about 70 to about 200 carbon atoms, from about 70 to about 150 carbon atoms, from about 70 to about 100 carbon atoms, from about 80 to about 200 carbon atoms, from about 80 to about 150 carbon atoms, or from about 80 to about 100 carbon atoms.

In other embodiments, one or more of the hydrophobic chains comprise a hydrophobic peptide, for example a poly(hydrophobic amino acid) (pHAA) such as poly(Phe), poly(Leu), poly(Val) and the like. The hydrophobicity of such chains may be measured by methods known in the art, for example the hydropathy index which quantifies the hydrophobic or hydrophilic nature of amino acid residues in a protein sequence. It will be appreciated that such methods may also be used to measure the hydrophobicity of amino acid-containing antigens, for example lipoproteins and lipoproteins.

When the antigen is hydrophobic or comprises at least one hydrophobic chain then it can be associated with or embedded in the lipid bilayer forming the walls or channels of the carrier. It has been surprisingly found that the incorporation of antigens even with multiple hydrophobic chains of significant length, such as those of trehalose dimycolate (cord factor or TDM) does not negatively impact upon the architecture of the carrier and still allows for successful delivery of the antigen.

In embodiments, the antigen is hydrophilic.

In embodiments, the antigen is contained in the lipid layer of the non-lamellar lyotropic liquid crystalline phase carrier.

In embodiments, the antigen is contained in the aqueous channels of the non-lamellar lyotropic liquid crystalline phase carrier.

In embodiments, the antigen is a subunit vaccine antigen.

In embodiments, the antigen is present at between about 0.1 to about 20 mol % relative to the total lipids in the content of the non-lamellar lyotropic liquid crystalline phase carrier. In certain examples the antigen may be present at between about 0.1 to about 10 mol %, between about 0.1 to about 9 mol %, between about 0.1 to about 8 mol %, between about 0.1 to about 7 mol %, between about 0.1 to about 6 mol %, between about 0.1 to about 5 mol %, between about 0.1 to about 4 mol %, between about 0.1 to about 3 mol %, between about 0.1 to about 2 mol %, between about 0.5 to about 10 mol %, between about 0.5 to about 9 mol %, between about 0.5 to about 8 mol %, between about 0.5 to about 7 mol %, between about 0.5 to about 6 mol %, between about 0.5 to about 5 mol %, between about 0.5 to about 4 mol %, between about 0.5 to about 3 mol %, or between about 0.5 to about 2 mol %.

In embodiments, the antigen is present at between 0.5 to 5 mol % relative to the total lipids in the content of the non-lamellar lyotropic liquid crystalline phase carrier.

In embodiments, the antigen is present at about 1 mol % relative to the total lipids in the content of the non-lamellar lyotropic liquid crystalline phase carrier.

The antigen may be selected from pathogenic antigens, tumour antigens, allergenic antigens or autoimmune self-antigens. In embodiments, the antigen is a pathogenic antigen. Such pathogenic antigens may be those derived from pathogenic organisms, in particular bacterial, viral or protozoological (multicellular) pathogenic organisms, which evoke an immunological reaction in a mammalian subject, such as a human. Pathogenic antigens may be surface or cell surface expressed antigens, for example proteins or portions or fragments thereof, located at least partly at the surface of the virus or the bacterial or protozoological organism.

Pathogenic antigens of interest may include those derived from one or more of: Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocysts hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, QD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo haemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, 0111 and 0104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rhodococcus spp, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sphingomonas spp, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In certain embodiments, relevant antigens may be derived from the pathogens selected from: Severe Acute Respiratory Syndrome (SARS), Severe Acute Respiratory Syndrome Coronavirus and Severe Acute Respiratory Syndrome Coronavirus 2 (SARS-CoV-1 and SARS-CoV-2), Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In some embodiments, the relevant pathogenic antigen may be selected from: Outer membrane protein A OmpA, biofilm associated protein Bap, transport protein MucK (Acinetobacter baumannii, Acinetobacter infections); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GalNAc lectin, protein CRT, 125 kDa immunodominant antigen, protein M17, adhesin ADH112, protein STIRP (Entamoeba histolytica, Amoebiasis); major surface proteins 1-5 (MSPla, MSPlb, MSP2, MSP3, MSP4, MSP5), type IV secretion system proteins (VirB2, VirB7, VirBll, VirD4) (Anaplasma genus, Anaplasmosis); protective Antigen PA, edema factor EF, lethal factor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa surface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp vl3, allergen Asp f3, major conidial surface protein rodlet A, protease Peplp, GPI-anchored protein Gellp, GPI-anchored protein Crflp (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 11C5, 21B4, P29, variant erythrocyte surface antigen VESA1, Apical Membrane Antigen 1 AMA-1 (Babesia genus, Babesiosis); hemolysin, enterotoxin C, PXOl-51, glycolate oxidase, ABC-transporter, penicillin-binding protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocysts hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin-binding protein A DbpA, decorin-binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); botulinum neurotoxins BoNT/A1, BoNT/A2, BoNT/A3, BoNT/B, BoNT/C, BoNT/D, BoNT/E, BoNT/F, BoNT/G, recombinant botulinum toxin F He domain FHc (Clostridium botulinum, botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-binding protein znuA, periplasmic immunogenic protein Bp26, 30S ribosomal protein Si2 RpsL, glyceraldehyde-3-phosphate dehydrogenase Gap, 25 kDa outer-membrane immunogenic protein precursor Omp25, invasion protein B lalB, trigger factor Tig, molecular chaperone DnaK, putative peptidyl-prolyl cis-trans isomerase SurA, lipoprotein Ompl9, outer membrane protein MotY Ompl6, conserved outer membrane protein D15, malate dehydrogenase Mdh, component of the Type-IV secretion system (T4SS) VirJ, lipoprotein of unknown function BABL_0187 (Brucella genus, Brucellosis); members of the ABC transporter family (Lo1C, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, Sapoviurus capsid protein VP1, protein Vp3, genome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspAl, protein FspA2(Campylobacter genus, Campylobacteriosis); glycolytic enzyme enolase, secreted aspartyl proteinases SAPI-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Pari, mucin-Associated Surface Proteins MPSP (Trypanosoma cruzi, Chagas Disease (American trypanosomiasis)); envelope glycoproteins (gB, gC, gE, gH, gl, gK, gL), (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp1O, Pmp11, Pmp12, Pmp13, Pmp14, Pmp15, Pmp16, Pmp17, Pmp18, Pmp19, Pmp20, Pmp21), (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis protein F TcpF, cholera enterotoxin subunit A, cholera enterotoxin subunit B, heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwpl3, cysteine protease Cwpl9, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (including rhinoviruses and coronaviruses, common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GalXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL105, glycoprotein gM, glycoprotein gN, glycoprotein H, glycoprotein B gB, protein UL83, protein UL94, protein UL99 (Cytomegalovirus (CMV), Cytomegalovirus infection); capsid protein C, pre-membrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoebafragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vQD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DltA, lipoprotein Hip, major outer membrane protein OMP, outer membrane protein OmpA2 (Haemophilus ducreyi, Chancroid); aspartyl protease 1 Pep1, phospholipase B PLB, alpha-mannosidase 1 AMN1, glucanosyltransferase GEL1, urease URE, peroxisomal matrix protein Pmp1, proline-rich antigen Pra, human T-cell reactive protein TcrP (Coccidioides immitis and Coccidioides posadasii, Coccidioidomycosis); allergen Tri r2, heat shock protein 60 Hsp60, fungal actin Act, antigen Tri r2, antigen Tri r4, antigen Tri t1, protein IV, glycerol-3-phosphate dehydrogenase Gpd1, osmosensor HwSholA, osmosensor HwSholB, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrane protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fms1 (EbpA(fm), Fms5 (EbpB(fm), Fms9 (EpbC(fm) and FmslO, protein EbpC(fm), 96 kDa immunoprotective glycoprotein GI (Enterococcus genus, Enterococcus infection); polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins see family, virulence proteins virB, tlyA, tlyC, parvulin-like protein Pip, pre-protein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gp1IO, glycoprotein gp42, glycoprotein gHgL, glycoprotein gB (Epstein-Barr Virus (EBV), Epstein-Barr Virus Infectious Mononucleosis); capsid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fas2, saposin-like protein-2 SAP-2, thioredoxin peroxidases TPx, Prx-1, Prx-2, cathepsin 1 cysteine proteinase CL3, protease cathepsin L CLI, phosphoglycerate kinase PGK, 27-kDa secretory protein, 60 kDa protein HSP35-alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component 1b, protein CPE1281, pyruvate ferredoxin oxidoreductase, elongation factor G EF-G, perfringolysin O Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); Clostridium species, Gas gangrene (Clostridial myonecrosis)); lipase A, lipase B, peroxidase Decl (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, α-giardin, α-8 giardin, α-1 giardin, β-giardin, cysteine proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, α-tubulin, beta-tubulin (Giardia intestinalis, Giardiasis); cyclophilin CyP, 24 kDa third-stage larvae protein GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein ompA, outer membrane transporter FetA, iron-repressed regulator MpeR (Neisseria gonorrhoeae, Gonorrhea); outer membrane protein C ompC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emml, M protein type 6 Emm6, immunoglobulin-binding protein 35 Sib35, Surface protein R28 Spr28, superoxide dismutase SOD, C5a peptidase ScpA, antigen I/II Ag/II, adhesin AspA, G-related a2-macroglobulin-binding protein GRAB, surface fibrillar protein M5 (Streptococcus pyogenes, Group A streptococcal infection); C protein β antigen, arginine deiminase proteins, adhesin BibA, 105 kDA protein BPS, surface antigens C, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein PI, outer membrane protein P2, outer membrane lipoprotein Pep, Lipoprotein E, outer membrane protein P4, fuculokinase FucK, [Cu,Zn]-superoxide dismutase SodC, protease HtrA, protein 0145, alpha-galactosylceramide (Haemophilus influenzae, Haemophilus influenzae infection); protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein GI, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein CpnlO, protein groES, heat shock protein HsplO, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, O-antigen, toxin-antigens Stx2B, toxin-antigen Stx1B, adhesion-antigen fragment Int28, protein EspA, protein EspB, Intimin, protein Tir, protein IntC300, protein Eae (Escherichia coli O157:H7, 0111 and 0104:H4, Hemolytic-uremic syndrome (HUS)); fusion protein F, polymerase L, protein W, protein C, phosphoprotein P, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein PiB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein; envelope glycoprotein E1 gp32 gp35, envelope glycoprotein E2 NS1 gp68 gp70, capsid protein C, core protein Core, polyprotein, antigen G, protein NS3, protein NS5A, (Hepatitis C Virus, Hepatitis C); large hepatitis delta antigen, small hepatitis delta antigen (Hepatitis D Virus, Hepatitis D); capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL 11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RSI), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); protein E, protein M, capsid protein C (Japanese encephalitis virus, Japanese encephalitis); RTX toxin, type IV pili, major pilus subunit PilA, regulatory transcription factors PIS and PilR, protein sigma 54, outer membrane proteins (Kingella kingae, Kingella kingae infection); prion protein (Kuru prion, Kuru); matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase Fl, beta-tubulin, heat shock protein 70 Hsp70, KMP-11, glycoprotein GP63, protein BT1, nucleoside hydrolase NH, cell surface protein B1, ribosomal protein P1-like protein PI, sterol 24-c-methy transferase SMT, LACK protein, histone HI, SPB1 protein, thiol specific antioxidant TSA, protein antigen STI1, signal peptidase SP, histone H2B, surface antigen PSA-2, cysteine proteinase b Cpb (Leishmania genus, Leishmaniasis); major membrane protein I, serine-rich antigen-45 kDa, 10 kDa chaperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membrane lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol-specific phospholipase C PLC (PicA, PlcB), 0-acetyltransferase Oat, ABC-transporter permease Im.G_1771, adhesion protein LAP, LAP receptor Hsp60, adhesin LapB, haemolysin listeriolysin OLLO, protein ActA, Internalin A InIA, protein InIB (Listeria monocytogenes, Listeriosis); protein Cox-2 (Wuchereria bancrofti and Brugia malayi, Lymphatic filariasis (Elephantiasis)); matrix protein Z, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbeta merozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, matrix protein VP40, envelope glycoprotein GP (Marburg virus, Marburg hemorrhagic fever (MHF)); hemagglutinin glycoprotein H; members of the ABC transporter family (LolC, OppA, and PotF), boaB coding protein (Burkholderia pseudomallei, Melioidosis (Whitmore's disease)); factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar to integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RP07 (Molluscum contagiosum virus (MCV), Molluscum contagiosum (MC)); matrix protein M, phosphoprotein PN, small hydrophobic protein SH, nucleoprotein N, protein V, fusion glycoprotein F, hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin PI, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500 coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); 43 kDa secreted glycoprotein, glycoprotein gpO, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cysteine protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, protein PM0305, formate dehydrogenase-N, protein PM0698, protein PM1422, DNA gyrase, lipoprotein PlpE, adhesive protein Cp39, heme acquisition system receptor HasR, 39 kDa capsular protein, iron-regulated OMP IROMP, outer membrane protein OmpA87, fimbrial protein Ptf, fimbrial subunit protein PtfA, transferrin binding protein Tbp1, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Pm, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Prn, protein Fim2, protein Fim3; “(Bordetella pertussis, Pertussis (Whooping cough)); “Fl capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YPO0612, heat shock protein 90 HtpG, putative sulfatase protein YdeN, outer-membrane lipoprotein carrier protein LolA, secretion chaperone YerA, putative lipoprotein YPO0420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla; “(Yersinia pestis, Plague); protein PhpA, surface adhesin PsaA, pneumolysin Ply, ATP-dependent protease CIp, lipoate-protein ligase LplA, cell wall surface anchored protein psrP, sortase SrtA, glutamyl-tRNA synthetase GltX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); protein Nfa1, exendin-3, secretory lipase, cathepsin B-like protease, cysteine protease, cathepsin, peroxiredoxin, protein Cry1Ac (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp1O, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein PI, heat shock protein B HspB, peptide ABC transporter, GTP-binding protein, protein IcmB, ribonuclease R, phosphatas SixA, protein DsbD, outer membrane protein TolC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L; matrix protein M, matrix protein M2-1, matrix protein M2-2, major surface glycoprotein G, non-structural protein 1 NS1, non-structural protein 2 NS2 (Respiratory syncytial virus (RSV), Respiratory syncytial virus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); intracytoplasmic protein D (Rickettsia akari, Rickettsialpox); envelope glycoprotein GP, non-structural protein NSS (Rift Valley fever virus, Rift Valley fever (RVF)); intracytoplasmic protein D (Rickettsia rickettsii, Rocky mountain spotted fever (RMSF)); non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, nonstructural glycoprotein 4 NS4, outer capsid protein VP4; (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis); protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASA1, glutathione S-transferases GST, cysteine protease, serine protease, apolipoprotein (Sarcoptes scabiei, Scabies); glutathione S-transferases GST, paramyosin, hemoglbinase SM32, major egg antigen, 14 kDa fatty acid-binding protein Sm14, major larval surface antigen P37, 22.6 kDa tegumental antigen, calpain CANP, triosephosphate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein CI, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A CifA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomyosin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein I Tpr1, repeat protein J TprJ, repeat protein K TprK, treponemal membrane protein A TmpA, lipoprotein, 15 kDa Tppl5, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MIC6, MIC7, MIC8), rhoptry protein Rop2, rhoptry proteins (Rop1, Rop2, Rop3, Rop4, Rop5, Rop6, Rop7, Rop16, Rjop17), protein SRI, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesin AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IglC1, intracellular growth locus IglC2, aminotransferase Wbt1, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen; (Ureaplasma urealyticum, Ureaplasma urealyticum infection); non-structural polyprotein, structural polyprotein, capsid protein CP, protein E1, protein E2, protein E3, protease PI, protease P2, protease P3 (Venezuelan equine encephalitis virus, Venezuelan equine encephalitis); protease NS3, protein AS2B, protein NS4A, protein NS4B, protein NS5 (Yellow fever virus, Yellow fever, West Nile virus, West Nile Fever); capsid protein CP, protein E1, protein E2, protein E3, protease P2 (Western equine encephalitis virus, Western equine encephalitis); putative Yop targeting protein YobB, effector protein YopD, effector protein YopE, protein YopH, effector protein YopJ, protein translocation protein YopK, effector protein YopT, protein YpkA, flagellar biosyntheses protein FihA, peptidase M48, potassium efflux system KefA, transcriptional regulatoer RovA, adhesin Ifp, translocator protein LcrV, protein PcrV, invasin Inv, outer membrane protein OmpF-like porin, adhesin YadA, protein kinase C, phospholipase CI, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspA1, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, protein PsaA (Yersinia enterocolitica, Yersiniosis), and lipopolysaccharide (LPS).

The relevant pathogenic antigen may also be selected from a lipid-based microbial antigen, for example an antigen capable of binding with Cluster of Differentiation 1 (CD1) molecules. Examples of such antigens include but are not limited to: diacylglycerol glycolipids such as α-glucosyldiacylcerol (αGlc-DAG-s2) (Streptococcus pneumoniae), α-galactosyldiacylglycerol (αGa1DAG) (Borrelia burgdorferi), and α-galactosyldiacylglycerols (BbGL-2c and BbGL-2f) (Borrelia burgdorferi); glycosphingolipids such as α-galactosylceramide (αGalCer), α-galactosylceramide Bacteroides fragilis (αGalCer_{Bf}) (Bacteroides fragilis), Agelasphin-9b (Agelas spp.), and α-galacturonosylceramide (GalA-GSL) (Sphingomonas spp.); phospholipids such as Phosphatidylglycerol (PG) (Listeria monocytogenes) and Phosphatidylglycerol (PI) (Listeria monocytogenes); mycoketides such as Phosphomycoketide (PM) (Mycobacterium tuberculosis) and Mannosyl-phosphomycoketide (MPM) (Mycobacterium tuberculosis); cholesterol glucosides such as αCAG (Helicobacter pylori); diacylated sulfoglycolipids such as Diacylsulfoglycolipid (Ac₂SGL) (Mycobacterium tuberculosis); mycolates such as Glucose monomycolate (GMM) (Mycobacterium tuberculosis), Glycerol monomycolate (Mycobacterium tuberculosis), Mycolic acid (Methoxy) (Mycobacterium tuberculosis), Glucose monomycolate-C54 (GMM-C54) (Nocardia spp.), and Glucose monomycolate-C32 (GMM-C32) (Rhodococcus spp.); lipopeptides such as dideoxymycobactin (Mycobacterium tuberculosis) and Mycobactin lipopeptide (Mycobacterium spp.); lipopeptidyphosphoglycans such as EhPIa (Entamoeba histolytica) and EhPIb (Entamoeba histolytica); lipophosphoglycans such as Phosphatidylinositolmannoside (PIM) (Mycobacterium tuberculosis) and lipoarabinomannan (LAM) (Mycobacterium tuberculosis); and mycolyl glycolipids such as trehalose 6,6′ dimycolate (TDM).

In embodiments wherein the infectious disease is influenza, the antigen may be derived from hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, or polymerase basic protein 2 (PB2) of an influenza virus or a fragment or variant thereof.

In certain examples, the antigen may be an antigen against one or more of a cancer, malaria, tuberculosis, campylobacter, influenza, rabies, RSV, pneumococcus and HBV.

In certain examples, the antigen may be selected from the group consisting of a BCG-Cell Wall extract, RTS, S/AS01 (Recombinant/Hybrid protein subunit), circumsporozoite protein (from malaria parasite) and HBsAg (hepatitis B virus), BCG-Subunit vaccine candidates+Mtb proteins and lipids, MTBVAC, GamTBvac, H56:IC31, ID93/GLA-SE, recombinant flagellin subunit vaccine, KRAS antigens, a haemagglutinin, G glycoprotein [NY-ESO-1, tyrosinase, MAGE-A3, TPTE], PCV-7, PCV-10, PCV-13, and HBsAg (also known as the Australia antigen).

In embodiments, the antigen is mycolic acid (or cord factor trehalose 6,6′ dimycolate).

It will be appreciated that the antigen of the non-lamellar lyotropic liquid crystalline phase carrier of the first aspect may be suitably selected depending on an infection, disease, disorder or condition to be treated in a subject, including the infections, diseases, disorders and conditions as described herein.

In a second aspect of the present disclosure, there is provided an immunogenic composition comprising a non-lamellar lyotropic liquid crystalline phase carrier of the first aspect and a pharmaceutically acceptable carrier, diluent and/or excipient.

As used herein, the term “immunogenic” will be understood to mean that the composition induces or generates an immune response. Suitably, the immune response is a protective immune response. By “protective immune response” is meant an immune response that is sufficient to prevent or at least reduce the severity or symptoms of an infection with, for example, a pathogenic organism. As used herein, “elicits an immune response” or “induces an immune response” indicates the ability or potential of the immunogenic composition to elicit or generate an immune response to an antigen upon administration of to the subject. As used herein, “immunize” and “immunization” refer to administering the immunogenic composition to elicit or potentiate a protective immune response to the antigen.

In certain examples, the immune response is or comprises a T-cell mediated immune response (i.e., a cell-mediated immune response) and/or a B-cell mediated immune response (i.e., a humoral immune response).

In embodiments, the immunogenic composition is a vaccine.

In certain examples, the immunogenic composition does not comprise or is substantially free of an adjuvant which is not a constituent part of the non-lamellar lyotropic liquid crystalline phase carrier itself.

In certain examples of the immunogenic composition, the components of the non-lamellar lyotropic liquid crystalline phase carrier other than the antigen do not or do not substantially induce or generate an immune response. That is, only the antigen non-lamellar lyotropic liquid crystalline phase carrier induces or generates the immune response. Advantageously, as shown in the Examples, the non-lamellar lyotropic liquid crystalline phase carrier is capable of acting solely as a carrier for presenting the antigen without itself eliciting an immune response.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

Preferably, the immunogenic composition is a liquid dispersion of the carriers of the first aspect. The liquid dispersion may be an aqueous dispersion.

The immunogenic compositions of the invention may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such immunogenic compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. The immunogenic compositions administered to a subject may be in the form of one or more dosage units, where for example, a tablet or injectable liquid volume may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000).

The immunogenic compositions may be useful for parenteral, topical, oral, or local administration, intramuscular administration, aerosol administration, or transdermal administration, for prophylactic or for therapeutic treatment. In one embodiment, the immunogenic composition of the second aspect is administered parenterally, such as intramuscularly, subcutaneously or intravenously. In some embodiments, the immunogenic composition of the second aspect is administered intramuscularly.

Formulation of the carriers of the first aspect to be administered will vary according to the route of administration and formulation (e.g., solution, emulsion, capsule) selected. An appropriate immunogenic composition comprising carriers of the first aspect to be administered can be prepared in a physiologically acceptable carrier. For solutions or emulsions, suitable pharmaceutical carriers include, for embodiment, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The immunogenic compositions can optionally contain pharmaceutically acceptable auxiliary substances as required to approximate physiological conditions such as pH adjusting and buffering agents and toxicity adjusting agents, for embodiment, sodium acetate, sodium chloride, potassium chloride, calcium chloride and sodium lactate. The carriers of the first aspect can be stored in the liquid stage or can be lyophilized for storage and reconstituted in a suitable carrier prior to use according to art-known lyophilization and reconstitution techniques.

When the immunogenic composition of the second aspect is a vaccine composition then the liquid carrier may be water, typically pyrogen-free water; isotonic saline or buffered (aqueous) solutions, e.g phosphate, citrate etc. buffered solutions. For injection of a vaccine composition, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, such as at least 3 mM of a potassium salt. In an embodiment, the sodium, calcium and, optionally, potassium salts may be present as their chlorides, iodides, or bromides, or in the form of their hydroxides, carbonates, hydrogen carbonates, or sulfates, etc. Non-limiting examples of sodium salts include e.g. NaCl, NaI, NaBr, Na2CO3, NaHCO3, Na2SO4, examples of the optional potassium salts include e.g. KCl, KI, KBr, K2CO3, KHCO3, K2SO4, and examples of calcium salts include e.g. CaCl2), CaI2, CaBr2, CaCO3, CaSO4, Ca(OH)2. Furthermore, organic anions of the aforementioned cations may be contained in the buffer. In certain embodiments, the buffer suitable for injection purposes, may contain salts selected from sodium chloride (NaCl), calcium chloride (CaCl2)) and optionally potassium chloride (KCl), wherein further anions may be present additional to the chlorides. In embodiments, the salts in the injection buffer are present in a concentration of at least 50 mM sodium chloride (NaCl), at least 3 mM potassium chloride (KCl) and at least 0.01 mM calcium chloride (CaCl2)). The injection buffer may be hypertonic, isotonic or hypotonic with reference to the specific reference medium.

In some embodiments of a vaccine composition, one or more compatible solid or liquid fillers or diluents or encapsulating compounds may be employed which are suitable for administration to a person. Pharmaceutically acceptable carriers, fillers and diluents will have sufficiently high purity and sufficiently low toxicity to make them suitable for administration to a subject. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; and alginic acid.

When the immunogenic composition of the second aspect is a vaccine composition it may further comprise one or more pharmaceutically acceptable adjuvants to enhance the immunostimulatory properties of the composition. The adjuvant may be any compound, which is suitable to support administration and delivery of the composition, and which may initiate or increase an immune response of the innate immune system, i.e., a non-specific immune response.

Such an adjuvant may be selected from any adjuvant known to a skilled person and suitable for the particular nature of the vaccine composition, i.e., for induction of a suitable immune response in a mammal. In embodiments, the adjuvant may be selected from the group consisting of: MF59® (squalene-water emulsion), TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(bl-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, AL); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thrl]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG polynucleotide sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/ILewis X, antimicrobial peptides, UC-1V150, RSV fusion protein, cdiGMP; and adjuvants suitable as antagonists including CGRP neuropeptide. In one preferred example the adjuvant may be the oil-in-water emulsion adjuvant MF59@, particularly if the vaccine is an influenza vaccine.

However, in embodiments the immunogenic composition of the second aspect a separate adjuvant may not be required as the carrier of the first aspect may, itself, induce a suitable innate immune response.

In some examples, the immunogenic composition further comprises a cell targeting ligand. To this end, lipid nanoparticles or vesicles of the immunogenic composition can be targeted to receptors on antigen presenting cells (APCs), for example, by placing ligands for cellular receptors of APCs on the surface of the particle (for example, mannosyl moieties or complement proteins such as C3d). For such examples, the immunogenic composition additionally comprises a cell targeting ligand at or on the surface of the lipid nanoparticle. The cell-targeting ligand facilitates the delivery of the immunogenic composition to an immune cell, such as an APC. In some particular examples, the immune cell is an APC, such as a dendritic cell and/or a macrophage. In some other examples, the immune cell comprises a mannose receptor or a C-lectin type receptor on its cell surface.

In a third aspect of the present disclosure, there is provided a method of delivering an antigen to a cell including the step of contacting the cell with the non-lamellar lyotropic liquid crystalline phase carrier of the first aspect, or the immunogenic composition of the second aspect.

In a fourth aspect of the present disclosure, there is provided a method of inducing an immune response in a subject including the step of administering an effective amount of the non-lamellar lyotropic liquid crystalline phase carrier of the first aspect, or the immunogenic composition of the second aspect, to the subject.

Suitable regimens for the administration of the immunogenic compositions disclosed herein are known in the art. The above compositions may be administered in a manner compatible with the dosage formulation, and in such amount as effective. The dose administered to a patient, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In some cases, as little as one dose of the immunogenic composition is needed, but under some circumstances, such as conditions of greater immune deficiency, a second, third or fourth dose may be given. Following an initial vaccination, subjects can receive one or several booster immunizations adequately spaced.

The immunogenic compositions disclosed herein may be given as a single dose. Alternatively, immunogenic compositions disclosed herein may be given in a multiple dose schedule. In one example, the multiple dose schedule consists of a series of two doses separated by an interval of about 1 month to about 2 months.

In another example, the multiple dose schedule consists of a series of two doses separated by an interval of about 1 month, or a series of two doses separated by an interval of about 2 months.

In a fifth aspect of the present disclosure, there is provided a method of preventing, treating or ameliorating an infection, disease, disorder or condition including the step of administering a therapeutically effective amount of a non-lamellar lyotropic liquid crystalline phase carrier of the first aspect, or the immunogenic composition of the second aspect, to a subject in need thereof to thereby prevent, treat or ameliorate the infection, disease, disorder or condition.

In a sixth aspect of the present disclosure, there is provided a non-lamellar lyotropic liquid crystalline phase carrier of the first aspect, or the immunogenic composition of the second aspect, for use in preventing, treating or ameliorating an infection, disease, disorder or condition.

In a seventh aspect of the present disclosure, there is provided a use of a non-lamellar lyotropic liquid crystalline phase carrier of the first aspect, or the immunogenic composition of the second aspect, in the manufacture of a medicament for the prevention, treatment or amelioration of an infection, disease, disorder or condition.

It will be appreciated that the infection, disease, disorder or condition to be treated will depend on the antigen of the non-lamellar lyotropic liquid crystalline phase carrier of the first aspect or the immunogenic composition of the second aspect.

In embodiments, the infection, disease, disorder or condition is a bacterial, protozoological, viral or fungal infection, including those described herein or caused by organisms described herein. In embodiments, the infection, disease, disorder or condition is a bacterial or viral infection. In embodiments, the bacterial infection is tuberculosis (TB). In embodiments, the viral infection is influenza.

In embodiments, the infection, disease, disorder or condition is a bacterial infection. In one embodiment, the infection, disease, disorder or condition is tuberculosis (TB).

In embodiments, the infection, disease, disorder or condition is caused by a pathogenic bacterial, protozoological, viral or fungal organism, including those described herein. In embodiments, the infection, disease, disorder or condition is caused by a pathogenic organism selected from Severe Acute Respiratory Syndrome (SARS), Severe Acute Respiratory Syndrome Coronavirus and Severe Acute Respiratory Syndrome Coronavirus 2 (SARS-CoV-1 and SARS-CoV-2), Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In embodiments, the infection, disease, disorder or condition is caused by a pathogenic bacterial or viral organism. In embodiments, the bacterial organism is selected from Staphylococcus aureus, Chlamydia trachomatis, and Mycobacterium tuberculosis. In one embodiments, the bacterial organism is Mycobacterium tuberculosis. In embodiments, the viral organism is selected from Severe Acute Respiratory Syndrome (SARS), Severe Acute Respiratory Syndrome Coronavirus and Severe Acute Respiratory Syndrome Coronavirus 2 (SARS-CoV-1 and SARS-CoV-2), Influenza virus, respiratory syncytial virus (RSV), Herpes simplex virus (HSV), human Papilloma virus (HPV), Human immunodeficiency virus (HIV), Dengue virus, Cytomegalovirus (CMV), Hepatitis B virus (HBV), Rabies virus, and Yellow Fever Virus.

In embodiments, the infection, disease, disorder or condition is caused by a pathogenic bacterial organism. In one embodiment, the infection, disease, disorder or condition is caused by Mycobacterium tuberculosis (MTB).

In embodiments, the infection, disease, disorder or condition is cancer.

In embodiments, the infection, disease, disorder or condition in selected from cancer, malaria, tuberculosis, campylobacter, influenza, rabies, RSV, pneumococcus and HBV.

In embodiments wherein the antigen is mycolic acid (or cord factor, trehalose 6,6′ dimycolate, TDM), then the infection, disease, disorder or condition is tuberculosis (TB) and/or is caused by Mycobacterium tuberculosis (MTB).

The following comments may apply, as appropriate, to any one or more of the third to seventh aspects.

Upon formulation, compositions of the present disclosure will be administered in a manner compatible with the dosage formulation and in such amount as is therapeutically/prophylactically effective. The dosage ranges for the administration of the carriers of the first aspect are those large enough to produce the desired effect. In one embodiment, the composition comprises an effective amount of the encapsulated or associated antigen. In one embodiment, the composition comprises a therapeutically effective amount of the antigen. In another embodiment, the composition comprises a prophylactically effective amount of the antigen.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Preparation methods for the above compounds and compositions are described further herein and/or are known in the art.

As generally used herein, the terms “administering” or “administration”, and the like, describe the introduction of the relevant carrier or immunogenic composition to a mammal such as by a particular route or vehicle. Routes of administration may include topical, parenteral and enteral which include oral, buccal, sub-lingual, nasal, anal, gastrointestinal, subcutaneous, intramuscular and intradermal routes of administration, although without limitation thereto.

By “treat”, “treatment” or treating” or “ameliorating” is meant administration of the relevant carrier or immunogenic composition to a subject to at least ameliorate, reduce or suppress existing signs or symptoms of the disease, disorder or condition experienced by the subject. to the extent that the medical condition is improved according to clinically acceptable standard(s).

By “prevent”, “preventing” or “preventative” is meant prophylactically administering the relevant carrier or immunogenic composition to a subject who does not exhibit signs or symptoms of a disease disorder or condition, but who is expected or anticipated to likely exhibit such signs or symptoms in the absence of prevention. Preventative treatment may at least lessen or partly ameliorate expected symptoms or signs.

As used herein, “effective amount” or “therapeutically effective amount” refers to the administration of an amount of the relevant carrier or immunogenic composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. An appropriate dosage or dosage regime can be ascertained through routine trial or based on current treatment regimes for the one or more actives being delivered via the particle of the first aspect.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment for a disease, disorder or condition as described herein. However, it will be understood that the aforementioned terms do not imply that symptoms are necessarily present.

In one embodiment, the subject is a human being vaccinated against a pathogenic organism.

The human patient may be a toddler (approximately 12 to 24 months), or young child (approximately 2 to 5 years). The compositions disclosed herein are also suitable for use with older children, adolescents and adults (e.g., aged 18 to 45 years, aged 18 to 50 years, aged 18 to 55 years, aged 18 to 60 years or 18 to 65 years).

In one example, the human patient is elderly. For example, the patient may be 50 years of age or older. In another example, the patient is 55 years of age or older. In another example, the patient is 60 years of age or older. In another example, the patient is 65 years of age or older. In another example, the patient is 70 years of age or older.

The patient to be treated with an immunogenic composition disclosed herein may be immunocompromised.

In some examples, the immunogenic composition may be administered concomitantly with a vaccine against another antigen, for example, influenza.

Optimal amounts of components for a particular vaccine can be ascertained by standard studies involving observation of appropriate immune responses in subjects. For example, the dosage for human vaccination is determined by extrapolation from animal studies to human data. In another embodiment, the dosage is determined empirically.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Example 1—Particle Formation

Monoolein (MO) was obtained from Nu-check-Prep, Inc (Minnesota, USA) with purity greater than 99% as determined by gas-liquid chromatography. Mycolic acid, Ethanol and Pluronic F127 were purchased from Sigma-Aldrich (NSW, Australia).

The dry powder of MO was dissolved in ethanol to prepare the stock solution at 200 mg/mL. TDM powder was dissolved in a solution of ethanol and chloroform (1:1 ratio) at 5 mg/mL stock solution. The MO-TDM complex was prepared by mixing the accurate stock solutions at a desired ratio in a glass vial. To evaporate the solvents, the lipid mixture was kept vacuum oven for overnight. Appropriate mol % of each lipid is calculated using the following equation:

$$mol \% MO = ( \frac{Total moles of MO}{Total moles of ( MO + TDM )} ) \times 100$$

Stock solution o pluronic F127 (0.5 wt %) was prepared in PBS, 1 ml of this polymer solution was added to the 50 mg of dry lipid mixture which is correspondent to 10% (w/w) of the total lipid. By using automated multiprobe sonicator all samples were dispersed on a high throughput platform. 80 μL (from 50 mg/mL stock) of dispersed nanoparticles were dispensed to a 96-well microplate, sealed with a microplate sealer and were kept at room temperature for further characterization.

### Example 2—Particle Morphology Characterisation

By using small and wide-angle X-ray scattering (SAXS/WAXS) beam, all nanocarrier structures were investigated at the Australian Synchrotron. A 1.033 Å (12.0 keV) X-ray wavelength was used with a 10¹³ photons/s flux. The attainable q-range was in the range 0.01-0.5 Λ-1 (where the scattering vector q=4π sin(θ)/λ, where λ represents the wavelength and θ represents the scattering angle). Using IDL-based ScatterBrain software (Kirby, N. M. et al. Journal of Applied Crystallography. 2013, 46, pages 1670-1680) the scattering images were integrated further. The nanoparticle samples were loaded in a standard polystyrene 96-well plate and positioned in the high-throughput plate holder, which was positioned 1 metre from a Decree-Pilatus 1-M detector, which recorded two-dimensional X-ray diffraction images (exposure time of 1 second). A recirculating water bath was used for temperature control, as previously described (Mulet, X. et al. Acc Chem Res. 2013, 46, pages 1497-1505).

Using IDL-based AXCESS software which has been developed at Imperial College London (Seddon, J. et al. Philos. Trans. Royal Soc. A Math Phys Eng Sci. 2006, 364, pages 2635-2655), SAXS data were analysed. The lattice parameters (LP) and different phase of each nano-formulations were also calculated using AXCESS software. As a standard calibrant (LP=58.38 Å) silver behenate was used in this software. Intensity vs q (scattering vector) of 1D plot was generated by conversion of 2D diffraction images acquired from the machine. On the basis of relative peak positions, the three cubic phases Q_{II}^{P}, Q_{II}^{D} and Q_{II}^{G} were designated. ‘a’ which is represent the lattice parameter was calculated from the ‘q-value’ of each peak using the equation:

$$a = \frac{2 {π ( {h}^{2} + {k}^{2} + {l}^{2} )}^{0.5}}{q}$$

where h, k, and l are the Miller indices. Each data point was run in duplicate from two different sets of samples for SAXS analysis.

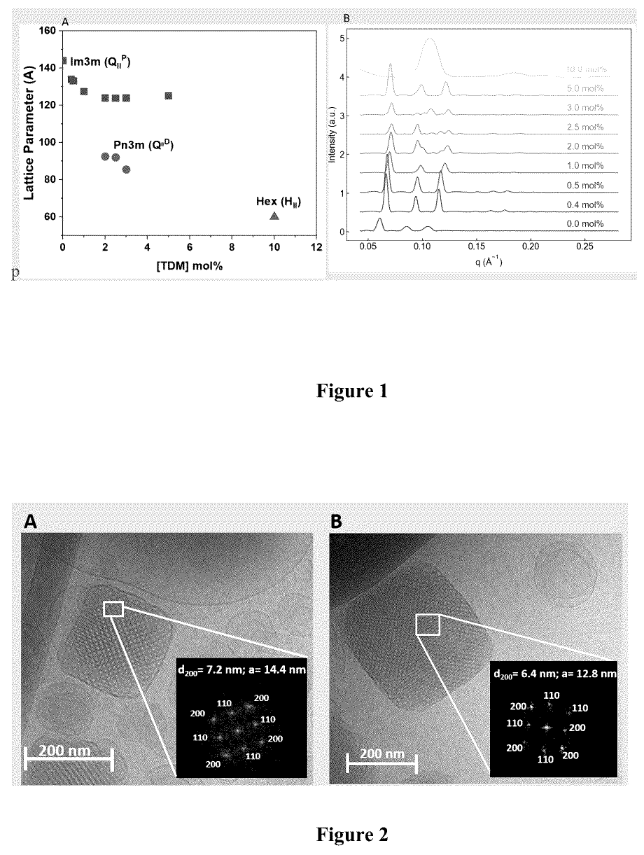

The structure of the bicontinuous cubic phases were characterised using SAXS for pure MO and for mixtures of MO doped with TDM in the concentration range from 0.4 mol % to 10 mol % (relative to MO) (FIG. 1). The phase adopted and the associated lattice parameter is plotted as a function of TDM concentration in FIG. 1A. Representative 1D SAXS patterns of intensity vs q are provided in FIG. 1B. Pure MO cubosomes adopted a primitive cubic (Q_{II}^{P}) symmetry at 25° C. with a typical lattice parameter ˜144 Å (FIG. 1 A) in agreement with the literature (Sarkar, S. et al. ACS Appl Mater Interfaces 13, pages 2336-2345, (2021)). The √2, √4 and √6 reflections of the Q_{II}^{P} cubic phase are observed in FIG. 1B. A decrease in lattice parameter of the Q_{II}^{P} phase was observed with increasing concentration of TDM up to 2.5 mol % when the lattice parameter appears to plateau. This suggests that the TDM is at least partially solubilised within the MO bilayer over this concentration range. For TDM concentrations ≤1 mol % Bragg peaks remain reasonably intense, sharp and well-defined, indicating the retention of long-range order within the cubic phase. At TDM concentrations ≥2 mol %, Bragg peaks become broad with a significant decrease in intensity, suggesting the encapsulated TDM has disrupted the Q_{II}^{P} long range order to some extent. From 2 mol % TDM the √2 and √3 peaks of a coexisting Q_{II}^{D} phase were observed. The lattice parameter of the Q_{II}^{D} phase was 92.4 Å at 2 mol %, decreasing slightly to 83.1 Å by 3 mol % TDM. The Bonnet ratio predicts the theoretical ratio of lattice parameters for two cubic phases coexisting at equilibrium and is 1.576 for Q_{II}^{P}/Q_{II}^{D} (Larsson; Current Opinion in Colloid & Interface Science 9, pages 365-369). The lattice parameter ratio Q_{II}^{P}/Q_{II}^{D} was 1.33, 1.35 and 1.50 at 2, 2.5 and 3 mol % TDM, respectively. At 5 mol % TDM a single Q_{II}^{P} phase is observed with a lattice parameter similar to that at 1 mol % TDM. However, by 10% TDM the system undergoes a transition from the Q_{II}^{P} phase to a Hu phase, suggesting that the inclusion of TDM at this high level within the MO promotes a phase transition to a more curved phase.

### Example 3—Particle Size Characterisation

The XRD patterns of the powder TDM (1 mg/mL), MO 1 mg/mL and mixture of MO (99 mol %)-TDM (1 mol %) in solid state, wherein samples were made on drop casting (that is, formation of a thin solid film by dropping a solution onto a flat surface followed by evaporation of the solution) of these materials on separate coverslips and allowed the solvent to evaporate. Data was recorded using a Bruker AXS D8 advance wide-angle X-ray diffraction instrument with a Cu Kα radiation source (1.54 Å).

### Differential Scanning Calorimetry (DSC)

In DSC, the differential heat flow to the aluminium pan carrying the sample and the empty reference pan are monitored by area thermocouples and a preheated purge gas was used to provide additional baseline stability. In this study 3 mg of dry powder of MO, TDM and MO-TDM (1%) mixture (in solid state) were weighed into aluminium DSC pans and the pans were sealed hermetically. Calorimetric analysis was performed on Mettler Toledo instrument over the temperature range from 4C to 60° C. with a heating rate 2° C./min within a nitrogen atmosphere. Instrument software were used for sample data acquisition and analysis.

### Fourier-Transform Infrared Spectroscopy (FTIR)

The FTIR spectra of TDM (1 mg/mL), MO 1 mg/mL and mixture of MO (99 mol %)-TDM (1 mol %) in solid state samples were recorded in Diffuse Reflectance Sampling accessory mode using a PerkinElmer D100 spectrophotometer with a resolution of 4 cm⁻¹. The extent of lipid mixing between MO and TDM was characterised for a MO-TDM (1 mol %) mixture using a combination of XRD, DSC and FT-IR.

### X-Ray Diffraction (XRD)

FIG. 4 shows the XRD patterns of the powder forms of TDM, MO and MO-TDM (1 mol %). Samples were prepared by drop casting as described above. Numerous sharp peaks in the XRD pattern of pure MO are consistent with the formation of a highly ordered crystalline structure upon the evaporation of solvent. Based on the known lattice parameter of the crystalline lamellar phase formed by pure MO (48.9 Å) the first observed Bragg peak at a 20 value of 5.64° (15.7 Å) is the (003) reflection. The formation of a crystalline structure is facilitated by H-bonding amongst the carboxylic acid groups in the MO headgroup.

In contrast, no observable Bragg peaks in the XRD pattern of TDM is consistent with an amorphous structure. A mixed system consisting of MO-TDM (1 mol %) displayed an XRD pattern similar to that of pure MO. However, the sharp Bragg peaks in the low angle region had shifted to higher angle consistent with a reduction in the lamellar d-spacing and indicating at least partial mixing of the two lipids.

### Differential Scanning Calorimetry (DSC)

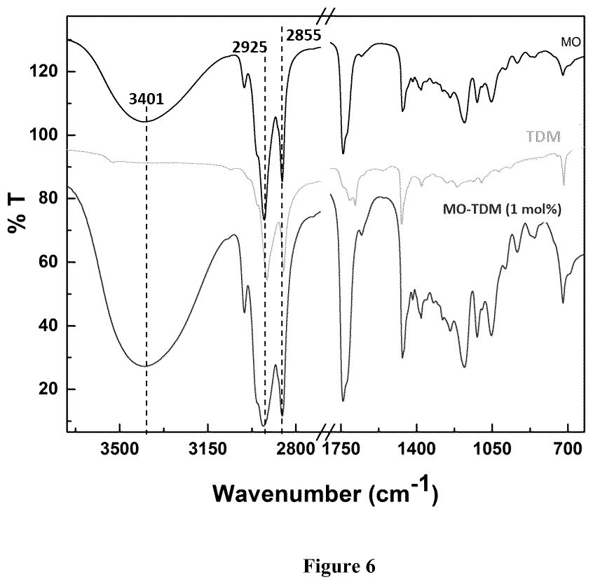

DSC scans of neat MO, TDM and MO-TDM (1 mol %) are presented in FIG. 6. MO exhibits a sharp endothermic peak at 33.5° C. that corresponding to its melting temperature. In contrast, the DSC scan of TDM did not show any sharp phase transition due to its amorphous nature. However, DSC scanning of TDM showed two thermal transitions at 34 and 53° C. The relatively high transition temperature (53° C.) of TDM is due to the melting of the mycolate chains and the methyl esters usually meet at lower temperature (34° C.). This is consistent with previous studies which report melting transitions of 33-35° C. and 53-65° C. for dehydrated neat MO and TDM, respectively. The DSC scan for MO-TDM (1 mol %) exhibited a single sharp peak near to the melting transition for MO but shifted to 32.25° C. and broadened slightly. A small decrease in melting point (33.5 to 32.25° C.) demonstrates mixing between the MO and TDM lipids, despite the very long chain length of TDM.

### Fourier-Transform Infrared Spectroscopy (FTIR)

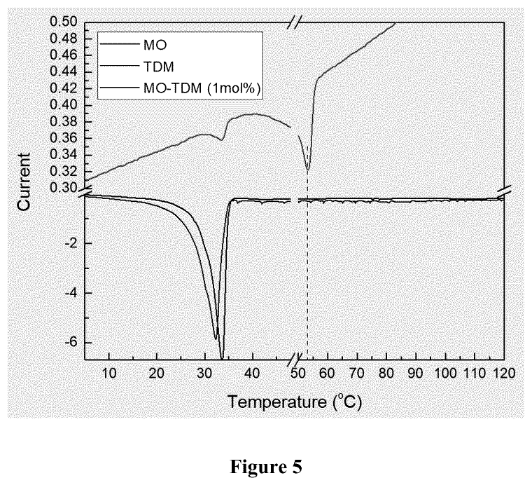

Both TDM and MO contain a carboxylic acid group in their headgroup region and both molecules can, therefore, potentially exhibit intermolecular hydrogen bonding. FTIR spectroscopy can provide information on the nature of the hydrogen bonding interactions between molecules. FTIR spectral data of MO, TDM and a mixture of MO-TDM (1 mol %) are presented in FIG. 5. MO exhibits an intense broad peak centred at 3401 cm⁻¹, characteristic of the —OH stretching frequency. The broad nature of this peak suggests intermolecular hydrogen bonding between the hydroxyl groups of MO. TDM didn't show any peaks characteristic of this O—H stretching frequency, suggesting no intermolecular hydrogen bonding between TDM molecules. Steric hindrance due to the extremely long hydrocarbon chains may inhibit the formation of hydrogen bonds in this case. In the case of MO-TDM (1 mol %) self-assembled structures, it was observed that the strong and broad band due to the —OH stretching frequency was slightly shifted; the intensity of this peak was also observed to decrease significantly. This suggests that the encapsulated TDM has affected the MO intermolecular H-bonding network providing further evidence of mixing between the two moieties. Furthermore, there were slight changes observed in the methylene symmetric (at 2925 cm-¹ to 2933 cm-¹) and antisymmetric (2855 cm-¹ to 2857 cm-¹) stretching frequencies. Also, the change in their peak width suggests packing of the TDM hydrocarbon chain within the MO hydrocarbon framework.

### Cryo-Transmission Electron Microscopy (Cryo-TEM)

Cryo-TEM samples were prepared using a FEI VITROBOT. The humidity and temperature were set up at 65% and 22° C. respectively on the device. 2 μL (50 mg/mL) of nanoparticle solution was added on top of a C-flat Holey Carbon grid and allowed 30 seconds for drying and blotted for 2 seconds. The sample loaded grid was then immediately immersed into liquid ethane solution. The processed carbon grids were stored in the liquid nitrogen container till taken to the cryo-holder (626 model) for further imaging. Transmission electron microscope (FEI Tecnai 12) is typically operated at 120 kV and was used for the samples imaging. The defocus level of 1.5-2 μm was used for each sample imaging at −190° C. The magnification range was between 35-100k under <900-1000 nm⁻² electron flow. ImageJ (NIH) software was used for Fast Fourier transform (FFT) analysis of each sample image.

### Nanocarrier Size Measurements

A Malvern Zetasizer Nano ZS (ZEN3600) instrument was used for measurement of the hydrodynamic diameter, polydispersity index, and ζ-potential of nano formulations. 20 μL (50 mg/mL) nano-formulations were added with 1 mL of Milli-Q H2O (18.2 MΩ cm). These diluted nano formulation solutions were transferred into a capillary cell (disposable) for particle size analysis. The sample measurements were counted by 12-13 runs in triplicate with 1.33 refractive index at 25° C. The zeta potential value was obtained by converting Electrostatic mobility numbers, using the Helmholtz-Smoluchowski equation for each sample.

The size, morphology and surface charge were characterised for MO cubosomes and MO-TDM cubosomes (1 mol %) using cryo-TEM and DLS. Representative cryo-TEM images of particles of MO and MO-TDM (1 mol %) are presented in FIG. 2.

In FIG. 2A, well-ordered typical cubic structure MO cubosomes are observed. The fast Fourier transforms (FFT) acquired from the optical diffractogram of the cryo-TEM images (shown as an inset in FIG. 2) confirmed the existence of internal cubic symmetry. For this Q_{II}^{P} phase, the distance between the d₂₀₀ planes is visible with a spacing of 7.2 nm, corresponding to a calculated lattice parameter of 14.4 nm. This value is in good agreement with the corresponding lattice parameter measured by SAXS analysis (14.5 nm). In FIG. 2B well-ordered cubosomes are still observed after addition of 1 mol % TDM, along with some aggregated particles. This is consistent with DLS results indicating an increase in particle size and increased polydispersity index (PDI) following encapsulation of 1 mol % TDM. The calculated lattice parameter value by FFT is 6.4 nm, in agreement with SAXS analysis (12.7 nm).

The ζ-potential of the pure MO cubosomes particles was measured as −12.4 mV (Table 1). Upon addition of 1 mol % TDM the zeta-potential became slightly less negative (−6.2 mV).

TABLE 1
Mean Hydrodynamic Diameter (MDD), Polydispersity Index
(PDI) and ζ-potential of MO and MO-TDM (1 mol %) cubosomes
(n = 2 ± SD).

| | MDD | | ζ-potential |
|---|---|---|---|
| Samples | (nm) | PDI | (mV) |
| MO | 210 ± 3 | 0.1 | −12.4 ± 1 |
| MO-TDM | 289 ± 2 | 0.3 | −6.2 ± 2 |
| (1 mol %) | | | |

### Example 4

THP-1, DU145 and Hela cell lines were purchased from ATCC. Phosphate-buffered saline was purchased from Thermo Fisher Scientific; MTS assay kit (Promega CellTiter 96 Aqueous One Solution) was purchased from Promega.

Cell Viability Evaluation Assessment with the Hela and THP-1 Cell Lines

The Hela and THP-1 monocyte cell lines were obtained from ATTCC Australia. The cell viability was determined by the number of viable cells corresponding to mitochondrial succinate dehydrogenase activity using the MTS assay kit. At a cell density 1×104 cells/mL (200 μL) cells (Hela and THP-1) were seeded in a 96 well plate. RPMI medium was used for cell culture and was supplemented with Fetal Bovine Serum (FBS; 10%) and a mixture of streptomycin (100 μg/mL) and penicillin (100 U/mL). Cells were incubated under a 5% CO2 environment at 37° C. for 24 hours. After 24 hours of incubation the culture medium was replaced with fresh medium. The cell confluency was observed at 72 hours. At a 20 μg/mL of MO, MO-TDM (1 mol %) lipid nano particles were added to cells and incubated for 72 hours. The cell culture medium was decanted and the treated cells were washed with PBS solution twice. 100 μL of fresh medium was added to the treated cells. 20 μL of MTS of MTS solution was added to each well and incubated for 4 hours with 5% CO2, at 37° C. A SpectraMax, Molecular Devices (microplate reader), was used for absorbance measurement at 490 nm. The MTS absorbance value of non-treated control cells was set to 100% and the absorbance values of nanoparticles treated cells were expressed as a percentage (%) of control cell viability. The cell viability experiments were carried out two times and each sample was kept in triplicate.

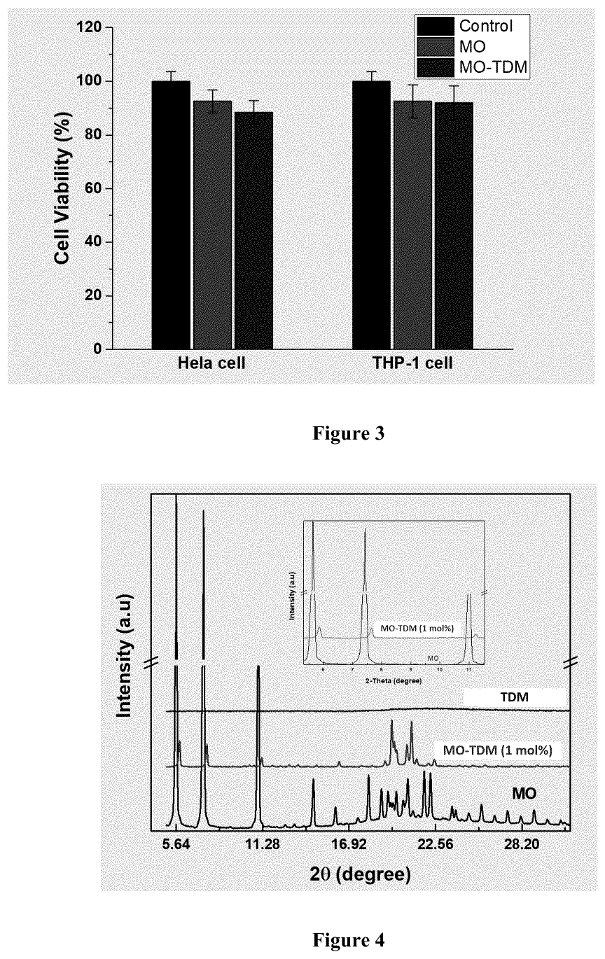

The cellular toxicity of MO and MO-TDM (1 mol %) cubosomes was assessed up to 72 hours incubation using an MTS assay at a total lipid concentration of 20 μg/mL (the concentration used for subsequent immunostimulatory experiments) against Hela cells (widely used for toxicity studies) and THP-1 (human monocyte) cell lines (FIG. 3). Stimulated THP-1 cells (macrophages) are the primary infection site of MTB infection in humans. MO cubosomes were non-toxic (>90% cell viability) against the Hela cell-line, consistent with previous research. A similar cell viability of >90% was observed for MO cubosomes against the THP-1 cell line. Addition of 1 mol % TDM did not affect the toxicity against the THP-1 cell-line. The cell viability decreased slightly against the Hela cell line for MO-TDM (1 mol %) cubosomes at the same lipid concentration (20 μg/mL). Numerous studies have investigated the in-vitro and in-vivo toxicity of MO based cubosomes although we note a significant variability in the measured toxicity due to use of different cell lines and assay methods.

### Example 5

### Bacterial Culture

Mtb (H37Rv) and BCG (Pasteur) were obtained from the American Type Culture Collection (ATCC). The Mtb and BCG strains were grown in Middlebrook 7H9 broth (Difco, Becton Dickinson) supplemented with 0.05% (v/v) Tween 80 and Middlebrook AODC Enrichment (Becton Dickinson) to mid-log phase (OD 600 nm=0.6-0.8) and reseeded in fresh media 7 days before infection, as previously described (Vermeulen; J Lipid Res 58, pages 709-718). Before being used for monocyte derived macrophages infection, bacterial cultures were washed three times in PBS and sonicated at 4 watts for 60 seconds using a sonicator (60 Sonic Dismembrator, Fisher Scientific) to prepare a uniform single-cell suspension of bacteria.

### Peripheral Blood Mononuclear Cell and Monocyte Isolation

Buffy coats from healthy donors were obtained. Peripheral blood mononuclear cells were isolated by dilution of blood in pyrogen-free phosphate buffer saline (PBS) and use of differential density centrifugation over Ficoll-Paque. The interphase layer was isolated, and cells were washed with cold PBS. The cells were further isolated and cultivated using plastic adherence and characterized by flow cytometry according to literature protocols. More than 90% of adherent cells were monocytes as determined by CD14 expression through flow cytometry (BD Biosciences). Monocytes were grown in Iscove's Dulbecco Modified Medium (IMDM) with 10% FBS for 6 days and harvested with 0.05% trypsin/l mM EDTA treatment (Life Technologies) for 5 min at 37° C. and re-suspended with fresh culture medium (IMDM+10% fetal bovine serum) for subsequent experiments in 6-well plates.

Trained Immunity Experiments with Human Monocytes

Human PBMCs (4×106 cells/well) were cultivated on flat-bottom 6-well plates. After washing with PBS, monocytes were incubated with culture medium only, as a negative control, or with TDM or MO or MO-TDM (1 mol %) for 18 hours. After incubation the cells were washed once with PBS and further incubated for 6 days in culture medium with 10% serum, and the medium was changed once at day 3. Cells were restimulated with IMDM (media control) or 10 ng/mL LPS on day 6. After 24 hours supernatants were collected and stored at −20° C. until cytokine measurement and cells were processed for chromatin immunoprecipitation (CHIP) qPCR.

### Cytokine Measurements

Quantification of cytokines produced by PBMCs was carried out by enzyme-linked immunosorbent assay (ELISA) using commercially available kits (Biolegend for human IL-6 and TNF-α). Supernatants were first filtered through a 0.22 μm filter (EMD Millipore, MA, USA) before they were titrated for cytokine levels according to the manufacturer's protocol.

Chromatin Immunoprecipitation (ChIP) qPCR

After the trained immunity experiment, human monocytes were fixed with 1% formaldehyde at a concentration of approximately 1 million cells/mL. Fixed cell preparations were sonicated (60 Sonic Dismembrator, Fisher Scientific) using 11 cycles of 30 seconds on and 30 seconds off. A total 25 μg of chromatin was incubated with dilution buffer containing protease inhibitor cocktail and 2 μg of H3K4me3 (Active Motif, Cat #39016) antibody and incubated overnight at 4° C. with rotation. Protein A/G magnetic beads were washed in dilution buffer with 0.15% SDS, 75 ng/μL single stranded herring sperm DNA and 0.1% BSA, added to the chromatin/antibody mix and rotated for 60 minutes at 4° C. Beads were washed with low salt, high salt, and LiCl wash buffers at 4° C. After washing, chromatin was eluted using 200 μL elution buffer for 20 minutes. Supernatant was collected, 4.8 μL 5M NaCl and 2 μl RNase A and incubated at 65° C. overnight. After that, samples were incubated with 2 μL proteinase K for 4 hours at 65° C. Finally, DNA samples were purified using QIAGEN MinElute PCR purification Kit and eluted in 20 μL elution buffer and subjected to qPCR analysis. Samples were analyzed using a % input method in which myoglobin was used as a negative control and H2B was used as a positive control for H3K4me3. The primers used for the analysis: Myoglobin; H2B; TNF-α and IL-6 were reported in an earlier study (Blok; European Journal of Clinical Microbiology & Infectious Diseases 38, 449-456).

### MTB Growth Assay in Macrophages

Macrophages were lysed with 0.05% SDS at different time-points post MTB infection with or without MO-TDM (1 mol %), TDM and MO. Lysates were plated at serial 10-fold dilutions in PBS using 7H11 Middlebrook agar plates (Difco Laboratories, Surrey, UK). The plates were incubated at 37° C. for 3 weeks before counting colony-forming units (CFUs). Data were expressed as log 10-CFUs per million macrophages.

### Ex Vivo MTB Ag85B Antigen Presentation to CD4 T Cells

Briefly, M. tuberculosis-infected MΦs were washed after a 4-hour infection and overlaid with the F9A6-CD4 T cell hybridoma which recognizes an Ag85B epitope in the context of human HLA-DR1. IL-2 secreted from hybridoma T cells or other cytokines secreted from M. tuberculosis-infected MΦs were determined using a sandwich ELISA kit (Biolegend).

### MO-TDM (1 Mol %) Nanoparticles Induce a Robust Pro-Inflammatory Cytokine Response in Macrophages

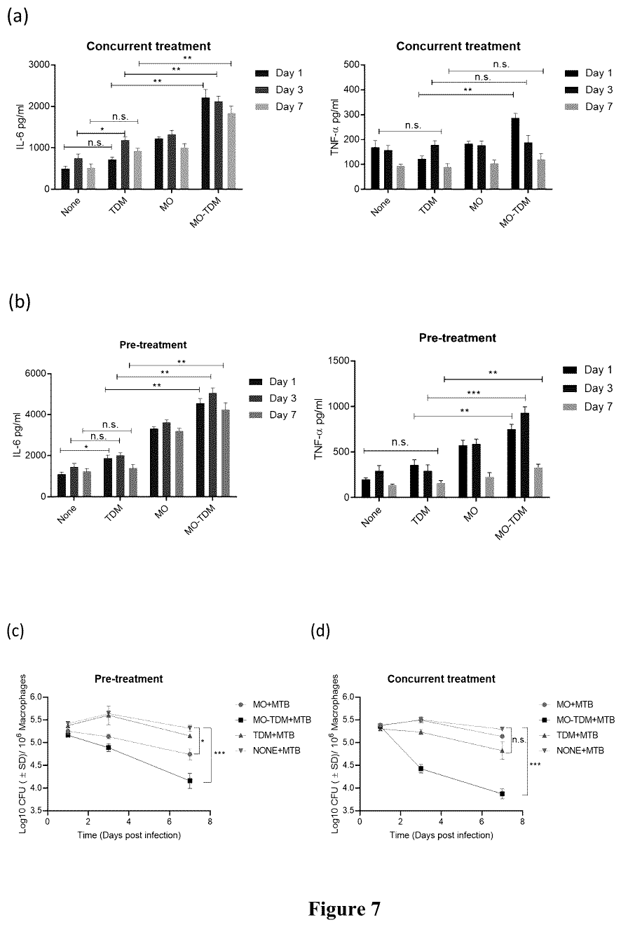

While the immunostimulatory effect of TDM has been studied, its effect when in a cubosomal nanoparticle formulation is unknown. To demonstrate a cubosomal preparation of TDM (1 mol %) with MO (99 mol %) could induce an altered immune response, as compared to the native TDM, the major proinflammatory cytokines IL-6 and TNF-α were examined after stimulation with MO, TDM, and MO-TDM (1 mol %), in untreated macrophages. Both TNF-α and IL-6 are known to be key cytokines involved in the generation of protective innate immune responses against MTB infection. Since macrophages are the primary host cells for MTB infection, these cytokines were examined in MTB infected macrophages after pre- and concurrent stimulation with MO, TDM, and MO-TDM (1 mol %). In pre-stimulation experiments, macrophages were treated with MO, TDM, and MO-TDM (1 mol %) 3 days prior to infection whereas in concurrent stimulation they were treated with TDM and MO-TDM (1 mol %) at the beginning of infection. During concurrent stimulation, a significantly increased secretion of both IL-6 and TNF-α was observed in macrophages that were treated with MO-TDM (1 mol %) as compared to untreated, MO or TDM treated macrophages (FIG. 7a). On the other hand, in pre-stimulated macrophages, not only MO-TDM (1 mol %) but MO alone was also able to induce elevated secretion of both IL-6 and TNF-α as compared to untreated and TDM treated macrophages (FIG. 7b). Nevertheless, the MO-TDM (1 mol %) preparation had a more prominent stimulatory effect on IL-6 and TNF-α secretion when compared with MO treated macrophages. In order to further examine if the increased pro-inflammatory immune response by macrophage induced by MO-TDM (1 mol %) could also affect the survival of MTB in macrophages, the viability of the pathogen was examined in a time dependent manner during concurrent as well as pre-stimulation with MO, TDM and MO-TDM (1 mol %). Nanocarrier (MO-TDM (1 mol %)) treated macrophages showed a significant reduction of more than 1 log 10 CFUs after 7 days of infection in concurrently stimulated as well as pre-stimulated macrophages (FIGS. 7c and 7d). Thus, similar to the increased inflammatory cytokine response, MO treated macrophages also induced a significant killing of M. tuberculosis in macrophages during pre-stimulation as well as concurrent stimulation. Overall, these results indicated that an MO-TDM (1 mol %) nanocarrier preparation induced a superior inflammatory immune response which also correlated with its enhanced antimycobacterial effect within macrophages.

### MO-TDM (1 Mol %) Induces Trained Immunity in Macrophages

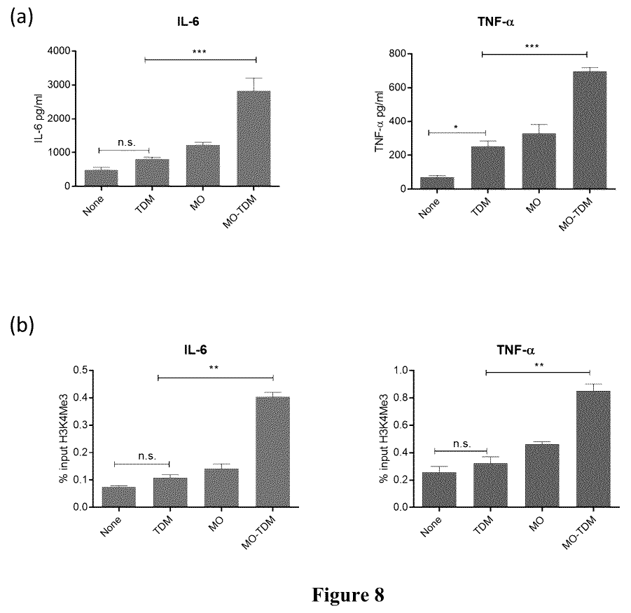

Although enhanced TNF-α secretion after TDM activation in macrophages is known, its further induction multi-fold by the MO-TDM (1 mol %) formulation was remarkable. Even more interesting was to observe the effect of an MO-TDM (1 mol %) preparation on IL-6 secretion by macrophages which has not been reported in any earlier studies so far with any of the TDM preparations. A paired stimulation of TNF-α and IL-6 together can be linked with trained immunity phenotype in macrophages, which indicated that MO-TDM (1 mol %) could be inducing trained immunity within macrophages. To assess the strength of trained immunity response by MO-TDM (1 mol %) cubosome, an in vitro model of primary human monocytes (PBMC) (Bekkering; Clin Vaccine Immunol 23, 926-933) was employed. Briefly, human PBMC derived adherent primary monocytes were primed with culture medium, only as a negative control, or MO or TDM or MO-TDM (1 mol %) for 18 hours and then rested for five days. On the 6th day, monocytes were stimulated with lipopolysaccharide (LPS) and the concentration IL-6 and TNF-α was measured. The same cells were used to study IL-6 and TNF-α promoter enrichment by H3K4me3 histone modification. Consistent induction of TNF-α and IL-6 in the human PBMCs was observed in response to the MO-TDM (1 mol %) acid cubosome that was significantly higher than that seen with the controls exposed monocytes (FIG. 8a).

The ability of the traditional BCG approach to elicit trained immunity has been correlated with changes in epigenetic markers that increase pro-inflammatory gene expression in the literature. H3K4me3 modification plays an important role in BCG-induced trained immunity as increased trimethylation of histone H3 at lysine 4 has been associated with an increased transcription of proinflammatory cytokine genes which represents the mechanism responsible for the long-term modulation of monocyte-derived cytokines. Therefore, it was of interest to see if the enhanced induction of TNF-α and IL-6 expression elicited by MO-TDM (1 mol %) cubosomes in comparison to the other controls is epigenetically mediated. Using chromatin immunoprecipitation-polymerase chain reaction (ChIP-PCR) assays, the activating histone methylation mark H3K4me3 present in the TNF-α and IL-6 promoters was quantified. It was observed that MO-TDM (1 mol %) cubosome induced a higher enrichment of H3K4me3 on the promoters of TNF-α and IL-6 in comparison to the other controls which suggests a stronger epigenetic training of monocytes (FIG. 8b). Upon re-stimulation with LPS at day 6, the abundance of the activating epigenetic mark was increased in MO-TDM (1 mol %) cubosome.

Epigenetic reprogramming in macrophages at the level of histone methylation and acetylation is associated with elevated expression of genes involved in glucose metabolism and reacquisition of pro-inflammatory traits of macrophages and M1 repolarization. This immunological process has been found to be effective in dealing with various infectious diseases and cancers. This has been shown to correlate with increased survival in tumor-bearing mice and in cancer patients. Therefore, a trained immunity inducing engineered MO-TDM (1 mol %) cubosome could be a fundamental mechanism to control tuberculosis infection in macrophages.

### MO-TDM (1 Mol %) Stimulated Macrophages Improved MHC-H Antigen Presentation to CD4T Cells

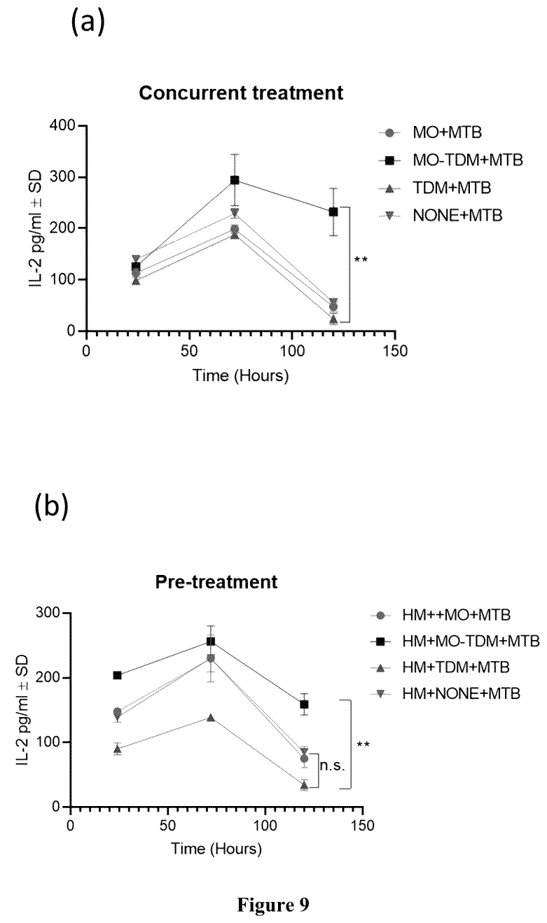

The micellar form of TDM, though known to induce an antibody mediated immune response, has been found to have a suppressive effect on cell mediated immunity in prior studies (Welsh; Tuberculosis 93, S3-S9). By inhibiting the phagosome lysosome fusion in macrophages, TDM not only protects the M. tuberculosis bacilli from intracellular killing but also reduces the priming of T cells due to reduced processing of Mycobacterial antigens in lysosomal compartments. Since increased killing of intracellular MTB in macrophages was observed when they were stimulated with a cubosomal MO-TDM (1 mol %) preparation, it indicated a possible increase in lysosmal processing of its antigens. Without wishing to be bound by theory, it is suggested that MO-TDM (1 mol %) carriers may prime the T cells better due to enhanced antigen processing and presentation of bacterial peptides. An in vitro antigen presentation assay using a CD4 hybridoma T cell that recognizes a specific epitope of Ag85B was adopted to determine the priming of T cells by macrophages that were stimulated with MO, TDM and MO-TDM (1 mol %). Secreted levels of IL-2 by hybridoma T cells upon overlay to M. tuberculosis infected macrophages with and without stimulation with MO, TDM and MO-TDM (1 mol %) were monitored in a time dependent manner (FIG. 9). During the concurrent activation antigen presentation to T cells, as indicated by secreted IL-2 levels, increased in macrophages stimulated with MO, TDM and MO-TDM (1 mol %) until day 3 but decreased sharply by day 5 post infection (FIG. 9a). From day 3 to day 5, the decrease in antigen presentation by MO-TDM (1 mol %) was significantly less as compared to TDM and MO stimulated macrophages which indicated a more beneficial sustained antigen processing and presentation by MO-TDM (1 mol %) treated macrophages. Though relatively less prominent, a similar observation was also noted in macrophages that were pre-stimulated with MO, TDM and MO-TDM (1 mol %), where MO-TDM (1 mol %) exhibited a more sustained antigen presentation as compared to TDM and MO treated macrophages (FIG. 9b). Since CD4 T cells process and present antigen through MHC-II pathways, which requires degradation of the bacteria/its proteins via lysosomal pathway, increased antigen presentation by MO-TDM (1 mol %) treated macrophages indicated that cubosomal nanocarriers of TDM with MO could overcome the inhibitory effect that TDM has on maturation of MTB containing phagosome, which led to its more efficient delivery to lysosomal compartments.

### Example 6

### In Vivo Prediction of MO-TDM (1 Mol %) Vaccine Efficacy Using Mathematical Modeling

A mechanistic mathematical model was developed to simulate and investigate the innate and adaptive immune response to the administered antigens (MO, TDM, and MO-TDM (1 mol %)) and MTB in vivo. The model is based on previous modeling works that involve the study of immune response to infectious agents, support the preclinical development of vaccines for pulmonary delivery, optimize vaccine dosing schedules and quantifying the in vivo pharmacmacokinetics of nanoparticle-based drug delivery systems. The model was calibrated for its innate immune response and antigen presentation process through the in vitro experimental data generated above (shown in FIGS. 8 and 9). The parsimonious model presented here incorporates the key processes related to antigen pharmacokinetics, cytokine-mediated innate immune response, and antigen presenting cell (APC)-induced adaptive immune response involving CD4+ T-cells and antibodies, following temporally separated injection of antigens and MTB into a virtual mouse body.

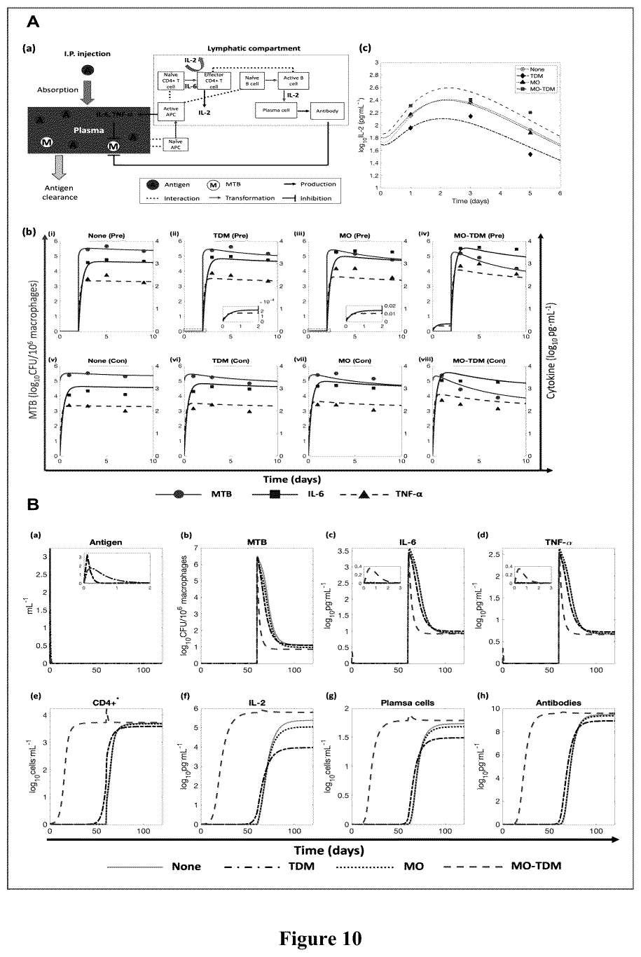

Specifically, the model comprises two compartments, i.e., a plasma and a lymphatic compartment, that communicate via the APCs and antibodies (FIG. 10A(a)). The antigen is injected intraperitoneally and is absorbed into systemic circulation (i.e., plasma compartment) following first order kinetics, where it is either cleared (renal and/or hepatobiliary excretion) or processed by naïve APCs, e.g., PBMCs. The interaction of antigens with the APCs in the plasma compartment activates the latter and they begin to secrete pro-inflammatory cytokines (IL-6 and TNF-α) to neutralize the live MTB (if any) and engage components of adaptive immunity. Upon activation, APCs migrate to the lymphatic compartment and interact with naïve CD4+ T-cells and B-cells to transform them into their effector forms, which leads to the production of IL-2 by the former and antibodies by the latter upon transformation into plasma cells. Of note, IL-6 promotes the activation of naïve CD4+ T-cells, while IL-2 promotes the proliferation of effector CD4+ T-cells (curved blue arrow in FIG. 10A(a)). Also, IL-2 induces the transformation of active B-cells into antibody-secreting plasma cells, which migrate to the plasma compartment to neutralize the live MTB (if any). Note that the same sequence of events occurs upon interaction of APCs with MTB.

The model parameters specific to mice were either known a priori from the literature or were estimated through non-linear least squares regression of the model to in vitro data relevant to innate immune response of the host (IL-6 and TNF-α-induced MTB death; FIG. 8), and antigen presentation to CD4+ T-cells (FIG. 9). To calibrate the model appropriately, the protocol used in the in vitro experiments was closely replicated with reduced forms of the model, i.e., Eqs. 2-4 for innate immune response characterization, and combined Eqs. 5 and 6 for antigen presentation characterization (details in Methods below).

In FIG. 10A (b) (i-iv), graphs show model fits of cytokine kinetics (squares, IL-6; triangles, TNF-α) in response to preincubation (day 0-2) of macrophages (i.e., APCs) with antigens (MO, TDM, MO-TDM (1 mol %)), followed by exposure to MTB (on day 2). In addition, the effect of cytokines on MTB population kinetics in the culture medium is also shown (circles). In contrast, the bottom row graphs (FIG. 10A (b) (v-viii) show cytokine and MTB kinetics upon concurrent administration of antigens and MTB (on day 0) to the culture medium containing macrophages. The numerical solution of the system of ODEs (Eqs. 2-4) replicating the above protocol and representing the interactions between antigens, macrophages, cytokines, and MTB was fit simultaneously to the experimental data under both the incubation conditions. As observed from the Pearson correlation analysis, the fits of Eqs. 2-4 to the MTB and cytokine kinetics are in good agreement with the in vitro data (R >0.9, P<0.0001), thereby providing confidence in the model-estimated parameter values.

As observed in the preincubation scenario (FIG. 10A (b) (i-iv))), cytokine production by macrophages in response to antigens is the highest upon incubation with MO-TDM (1 mol %) (FIG. 10A (b) (iv)), followed by MO (FIG. 10A (b) (iii)), and then TDM (FIG. 10 A (b) (ii)), as observed by the kinetics during the first two days with no MTB in the culture. As expected, the Michaelis-Menten constant k_{Ag} (indicative of the potency of the antigens to trigger cytokine production by macrophages) thus obtained is the lowest for the MO-TDM (1 mol %) scenario, suggesting the highest potency for the MO-TDM (1 mol %) antigen. The k_{Ag} for MO lies in between TDM and MO-TDM.

Upon the introduction of MTB in the culture on day 2 (FIG. 10A (b), (i-iv), cytokine levels continue to follow the same trend as before, resulting in a more rapid decline in MTB population in the MO-TDM (1 mol %) case, followed by MO, and TDM, in comparison to the no-antigen scenario. This may suggest antigen-induced priming of macrophages leading to enhanced cytokine production upon MTB exposure, which may cause an enhancement in cytokine-induced inhibition of MTB and is the most effective for MO-TDM (1 mol %). A similar trend is observed in the concurrent administration scenario (FIG. 10A (b) (v-viii), where concurrent administration causes almost the same enhancement in antigen-induced cytokine production and MTB decline as preincubation, which may indicate that the presence of antigens enhances the response of macrophages towards MTB.

Further, the antigen presentation by macrophages to CD4+ T-cells was characterized to obtain the kinetic parameters associated with IL-2 production by effector CD4+ T-cells upon completion of antigen presentation. As shown in FIG. 10A (c), the model fits (Eq. 9) are in good agreement with the experimental data, and it was observed that priming of macrophages with MO-TDM (1 mol %) leads to the most effective antigen presentation.

### Model Predictions

Following model calibration with experimental data and estimation of unknown parameters, the complete model was used to study the innate and adaptive immune response to intraperitoneal administration of antigens in mice (on day 0), followed by rechallenge with MTB on day 60. The plasma pharmacokinetics of antigen following intraperitoneal injection shows comparable behavior between the small molecule TDM and nanoparticulate antigens (MO, MO-TDM (1 mol %)), which can be expected due to the high renal excretion of the former and high hepatobiliary clearance of the latter, primarily driven by their respective sizes (FIG. 10B (a, inset)).

In response to the antigenic injection on day 0, an innate response in the form of IL-6 and TNF-α cytokines is mounted primarily in the MO-TDM (1 mol %) scenario that lasts up to −2 days post injection, but is less significant with the other two antigens (FIG. 10B (c), and (d), insets)). As a result, IL-6 induces the activation of CD4+ T-cells through antigen presentation, which is stronger in the MO-TDM (1 mol %) scenario, but weak in the TDM case, and it was forced to be absent in the empty cubosome (MO) scenario. Note: adaptive immunity against MTB can only be generated by vaccines carrying an MTB-specific antigen (e.g., MO-TDM (1 mol %) or TDM, but not MO).

Further, the model simulations predict that in an in vivo setting, vaccination with MO-TDM (1 mol %) antigen will lead to the production of MTB-specific, IL-2 secreting, effector CD4+ T-cells (FIG. 10B), which can self-sustain due to autocrine signaling of IL-2 (FIG. 10B (f)), and lead to the transformation of active B-cells into antibody-secreting plasma cells (FIG. 10B (g)). Thus, upon exposure to MTB (e.g., at day 60 in the current simulation, FIG. 10B (b)), the plasma compartment already contains MTB-specific effector CD4+ T-cells (FIG. 10B (e)) and antibodies (FIG. 10B (h)), where the latter are readily available for MTB neutralization, thereby bringing down the bacterial load by ˜5 orders of magnitude within −10 days of infection (FIG. 10B (b)). In contrast, vaccination with TDM does not produce significant effector CD4+ T-cells and antibodies, primarily due to its weak efficacy in inducing antigen presentation (FIG. 10A (c)). Thus, unlike MO-TDM (1 mol %), the TDM vaccine acts like the empty cubosome (MO) or no vaccine scenario and is primarily driven by the freshly mounted innate and adaptive immune response to MTB infection, i.e., no significant memory of prior exposure to the antigen is retained in the system. Thus, it takes up to ˜10 times longer to cause the same amount of MTB load reduction as the MO-TDM (1 mol %) case.

It is highlighted that the model developed here is a simplified representation of a highly complex and dynamic immune response involving numerous players. However, the primary aim of the model was to showcase the relative response of the immune system to various antigens investigated in this study, and thus the application of the same model to every antigen justifies its applicability in unraveling insights obtained from the numerical simulations. Additionally, in the absence of in vivo data, one-compartment pharmacokinetics has been assumed for all the antigens in the pharmacokinetic component of the model; future improvements may therefore be necessary for a more accurate representation of the pharmacokinetics, especially inclusion of the physicochemical properties of the antigens (e.g., size, zeta potential, shape, solubility, permeability, lipophilicity).

In summary, experiments discussed herein demonstrate the ability of MO-TDM cubosomes to induce a robust innate and adaptive response in vitro. The present mathematical modelling of the immune response to antigens further predicts the efficacy of MO-TDM to induce a potent innate and humoral immune response that is protective against simulated MTB rechallenge in vivo.

### Mathematical Model Equations

As given below, the model was formulated as a system of ordinary differential equations (ODEs) that describe the kinetics of MTB burden in response to the key innate and adaptive immunity variables evoked during exposure to the antigen and MTB.

### Antigen Concentration in Plasma (Ag(t)

For in vivo simulations, the administration of the antigen was modelled via the intraperitoneal route, such that the antigen is absorbed into systemic circulation at a rate k_{abs} and is cleared from systemic circulation at a rate kₑₓ.

$$\frac{dAg ( t )}{dt} = {\overbrace{{k}_{abs} \cdot \frac{{Ag}_{0}}{{V}_{p}} \cdot {e}^{- {k}_{abs} \cdot t}}}^{Absorption} - {\overbrace{{k}_{ex} \cdot Ag ( t )}}^{Clearance} , Ag ( 0 ) = 0 \qquad ( 1 )$$

where, Ag₀ is the administered dose of the antigen and Vₚ is the volume of the plasma compartment. Note that to simulate the in vitro experiments, it was assumed there was a constant level of antigen (=1) in the culture medium.

### MTB Density in Plasma (M(t))

The immune response to MTB infection involves both innate and adaptive components. The effects of IL-6, TNF-α, and antibodies in neutralizing MTB were included in the current model.

$$\frac{dM ( t )}{dt} = {\overbrace{γ \cdot M ( t )}}^{growth} - {\overbrace{{δ}_{cy} \cdot ( \frac{IL 6 ( t ) + TNF α ( t )}{{k}_{cy} + IL 6 ( t ) + TNF α ( t )} ) \cdot M ( t )}}^{Cytokine - induced death} - {\overbrace{{δ}_{AB} \cdot Ab ( t ) \cdot M ( t )}}^{Antibody - induced death} , \qquad ( 2 )$$

$$M ( 0 ) = \{ \begin{matrix}0 , t = 0 \\ {M}_{0} , t = i\end{matrix}$$

where, γ is the growth rate of MTB, δ_{cy} is the death rate of MTB induced by pro-inflammatory cytokines (IL-6 and TNF-α); k_{cy}, is the Michaelis-Menten constant of cytokines (IL-6, TNF-α, IL-2) to induce their biological effects; IL6(t) and TNFα(t) are the plasma concentrations of IL-6 and TNF-α, respectively; δ_{Ab} is the death rate of MTB by antibody-mediated phagocytosis; Ab(t) is the plasma concentration of antibodies; M₀ is the MTB load introduced into plasma at time i days to study immune response to rechallenge with MTB after an initial dose of antigen given as a vaccine on day 0.

### IL-6 Concentration in Plasma (IL6(t))

Cytokine production by macrophages is regulated by the concentration of the antigen or MTB in the plasma. This process was modelled using Michaelis-Menten kinetics.

$$\frac{dIL 6 ( t )}{dt} = {\overbrace{{p}_{IL 6} \cdot ( \frac{Ag ( t )}{{k}_{Ag} + Ag ( t )} + \frac{M ( t )}{{k}_{M} + M ( t )} )}}^{Production} - {\overbrace{{δ}_{IL 6} \cdot IL 6 ( t )}}^{Degradation} , IL 6 ( 0 ) = 0 \qquad ( 3 )$$

where, p_{IL6} and δ_{IL6} are the production and degradation rates of IL-6, respectively; k_{Ag} and k_{M} are the Michaelis-Menten constants of antigen and MTB effects on cytokine production by macrophages, respectively.

### TNF-α Concentration in Plasma (TNFα(t))

The TNF-α concentration in plasma_was measured.

$$\frac{dTNF α ( t )}{dt} = {\overbrace{{p}_{T} \cdot ( \frac{Ag ( t )}{{k}_{Ag} + Ag ( t )} + \frac{M ( t )}{{k}_{M} + M ( t )} )}}^{Production} - {\overbrace{{δ}_{TNF α} \cdot TNF α ( t )}}^{Degradation} , \qquad ( 4 )$$

$$TNF α ( 0 ) = 0$$

where, p_{T} and δ_{TNFα} are the production and degradation rates of TNF-α, respectively.

### Effector CD4+ T-Cell Concentration in Lymphatic Compartment (CD4*(t))

The population of effector CD4+ T-cells is governed by the activation of naïve CD4+ T-cells upon interaction with active APCs, mediated by IL-6 (first term of equation). Upon activation, CD4+ T-cells proliferate, and this process is promoted by IL-2 (second term of equation). Note that since active APCs are not explicitly modelled, the antigen and MTB concentration are used as a surrogate for active APCs in the first term of the equation below. Also, the population of effector CD4+ T-cells is limited by a carrying capacity to avoid unrealistic overpopulation due to proliferation.

$$\frac{dCD {4}^{*} ( t )}{dt} = {\overbrace{{T}_{CD 4} \cdot CD {4}_{0} \cdot {e}^{- {T}_{{CD}_{4}} \cdot t} \cdot \frac{IL 6 ( t )}{{k}_{cy} + IL 6 ( t )} \cdot ( \frac{Ag ( t )}{{k}_{Ag} + Ag ( t )} + \frac{M ( t )}{{k}_{M} + M ( t )} )}}^{Transformation} + {\overbrace{{p}_{CD 4} \cdot ( 1 + \frac{IL 2 ( t )}{{k}_{cy} + IL 2 ( t )} ) \cdot CD {4}^{*} ( t ) \cdot ( 1 - \frac{CD {4}^{*} ( t )}{\overline{CD 4}} )}}^{Proliferation} - {\overbrace{{δ}_{CD 4} \cdot CD {4}^{*} ( t )}}^{Death} , \qquad ( 5 )$$

$$CD {4}^{*} ( 0 ) = 0$$

where, T_{CD4} is the transformation rate of naïve CD4+ T-cells into their effector form; CD4₀ is the baseline population density of naïve CD4+ T-cells; p_{CD4} is the production rate of effector CD4+ T-cells, IL2(t) is the concentration of IL-2 in the lymphatic compartment; C̅D̅4̅ is the carrying capacity of effector CD4+ T-cells, and δ_{CD4} is the death rate of effector CD4+ T-cells.

### IL-2 Concentration in Lymphatic Compartment (IL2(t))

Upon interaction with antigen or MTB, effector CD4+ T-cells secrete IL-2 as a first order process, which undergoes degradation as a first order process as well.

$$\frac{dIL 2 ( t )}{dt} = {\overbrace{{p}_{IL 2} \cdot CD {4}^{*} ( t )}}^{Production} - {\overbrace{{δ}_{IL 2} \cdot IL 2 ( t )}}^{Degradation} , IL 2 ( 0 ) = 0 \qquad ( 6 )$$

where, p_{IL2} and δ_{IL2} are the production and degradation rates of IL-2, respectively.

### Plasma Cell Concentration in Lymphatic Compartment (Pl(t))

The process of transformation of naïve B-cells into their effector form, followed by the differentiation of the latter into plasma cells (under the influence of effector CD4+ T-cells and IL-2) has been simplified to obtain the kinetics of plasma cell population density by only explicitly incorporating the second step in the model (assuming it to be the rate limiting step).

$$\frac{dPI ( t )}{dt} = {\overbrace{{T}_{PI} \cdot \frac{IL 2 ( t )}{{k}_{cy} + IL 2 ( t )} \cdot CD {4}^{*} ( t )}}^{Transformation} - {\overbrace{{δ}_{PI} \cdot PI ( t )}}^{Death} , PI ( 0 ) = 0 \qquad ( 7 )$$

where, T_{Pl} and δ_{Pl} are the transformation and death rates of plasma cells, respectively.

### Antibody Concentration in Plasma (Ab(t1)

Antibody concentration kinetics is dependent on the production of antibodies by plasma cells, and clearance from the body.

$$\frac{dAb ( t )}{dt} = {\overbrace{{p}_{Ab} \cdot PI ( t ) \cdot ( 1 - \frac{AB ( t )}{\overline{AB}} )}}^{Production} - {\overbrace{CI \cdot Ab ( t )}}^{Clearance} , Ab ( 0 ) = 0 \qquad ( 8 )$$

where, p_{Ab} and Cl is the production and clearance rate of antibodies, respectively. A̅b̅ is the carrying capacity of antibodies in the body.

### Mathematical Model Calibration

Non-linear least squares regression was performed to fit the model to in vitro data (shown in FIG. 10) to calibrate the parameters associated with cytokine-induced MTB death and antigen presentation by APCs to CD4+ T-cells. For the first part of model calibration, Eqs. 2-4 were fit to the data shown in FIG. 10A with the simplification that antibody-induced death of MTB was ignored in Eq. 2, given the in vitro nature of the experiments. For the second part, the following equation was fit to the data given in FIG. 10B to estimate the unknown parameters:

$$\frac{dIL 2 ( t )}{dt} = {p}_{IL 2} \cdot ( \frac{{T}_{CD 4} \cdot CD {4}_{0} \cdot {e}^{- {δ}_{CD 4} \cdot t}}{{δ}_{CD 4} - {T}_{CD 4}} ) \cdot ( {e}^{( {δ}_{CD 4} - {T}_{CD 4} ) \cdot t} - 1 ) - {δ}_{IL 2} \cdot IL 2 ( t ) . \qquad ( 9 )$$

$$IL 2 ( 0 ) \neq 0$$

The above equation was obtained by combining Eqs. 5 and 6, while only including transformation and death processes in Eq. 5 and obtaining its closed-form solution to substitute in Eq. 6. Pearson correlation was performed to evaluate the goodness of fit of the model to experimental data. All analyses were performed in MATLAB R2018a

### Example 7—In Vivo Immunogenicity Experiments

Immunogenicity experiments in mice were conducted using ovalbumin (OVA) as a model antigen. The following nanoparticle carriers were assessed, with the polystyrene nanoparticle carriers serving as positive controls:
cubosomes; composition: monoolein (MO) and pluronic F127 in Milli Q water (at 10% w/w relative to the total lipid amount)
cationic cubosomes (or modified cubosomes); composition: dioleoyl-3-trimethylammonium propane (DOTAP, 1 mol %), monoolein (MO), pluronic F127 in Milli Q water (at 10% w/w relative to the total lipid amount)
polystyrene nanoparticles with an average particle size of 50 nm or 500 nm (PSNP 50 nm and PSNP 500 nm, respectively).

The following groups of mice were assessed (4 mice per group): Naïve, cubosomes-OVA, modified cubosomes-OVA, PSNPs-OVA 50 nm and PSNPs-OVA 500 nm. Experimental timeline: Day −1: pre-bleed; Day 0: first immunisation, intradermal at base of tail; Day 13: first bleed; Day 14: second immunisation, intradermal at base of tail; Day 28: cull and collect blood and spleens for ELISA and ELISpot assays. 50 μg of antigen was injected per mouse.

ELISpot results—IFNg-OVA: Splenocytes from immunised mice were co-cultured with whole OVA, the CD4 T cell epitope OVA-Helper, and the CD8 T cell epitope SIINFEKL, and assessed for IFNg cytokine responses by ELISpot assay. PSNPs-OVA 50 nm and 500 nm induced high responses for all antigens. PSNPs-OVA are a known inducer of antigen specific IFNg secretion from T cells, especially with 50 nm PSNPs, so the high responses to all antigens were expected. Cubosomes-OVA and modified cubosomes-OVA were shown not to elicit IFNg secretion compared to background.

ELISpot results—IL-4 OVA: Splenocytes from immunised mice were co-cultured with whole OVA and the CD4 T cell epitope OVA-Helper, and assessed for IL-4 cytokine responses by ELISpot assay. PSNPs-OVA 50 nm and 500 nm were shown to induce variable responses for all antigens, with PSNPs-OVA 50 nm inducing a moderate to high response to OVA-Helper. Cubosomes-OVA and modified cubosomes-OVA were shown not to elicit IL-4 secretion compared to background.

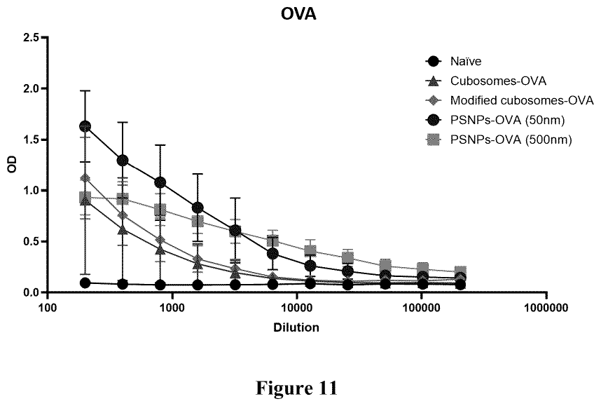

ELISA final bleed results: Serum was harvested from final bleeds of twice immunised mice and antigen specific OVA IgG antibodies were assessed using ELISA assay. Serum was serially diluted 1 in 2 starting at 1 in 200 dilution. FIG. 11 shows the antibody responses measured as optical density readings for each dilution and an average per group calculated. Naïve mice had low OVA-specific antibody responses, whereas the cubosomes-OVA, modified cubosomes-OVA and PSNPs-OVA groups were all shown to induce OVA specific antibody responses.

The ELISpot and ELISA assay results suggest that cubosomes-OVA and modified cubosomes-OVA may be eliciting B-cell mediated immune response. In particular, these formulations were not shown to induce IFNg or IL-4 secretion in the ELISpot assays, but were shown to induce OVA-specific antibody responses in the ELISA assay.

This mechanism was further investigated in IgG subtype specific ELISA assays. Immunoglobulin G (IgG) antibodies are secreted by B cells. The following subclasses of IgG were assessed using the protocol described above: IgG1, IgG2a, IgG2b and IgG3. Results: IgG1: cubosomes-OVA, modified cubosomes-OVA and PSNPs-OVA groups were all shown to induce OVA specific IgG1 antibody responses; IgG2a: all formulations were shown to elicit low OVA-specific antibody responses; IgG2b: modified cubosomes-OVA and PSNPs-OVA 50 nm were shown to induce higher OVA specific IgG2b antibody responses compared to cubosomes-OVA and PSNPs-OVA 500 nm; IgG3: all formulations were shown to elicit low OVA-specific antibody responses. Naïve mice had low OVA-specific antibody responses in all assays.

The above results provide an indication that cubosomes-OVA and modified cubosomes-OVA may be inducing B-cell mediated immune response. The OVA specific antibody responses also suggest that it is the OVA antigen that is raising the immune response, with the cubosomes and modified cubosomes solely acting as carriers for presenting the antigen and not themselves inducing an immune response.

1. A non-lamellar lyotropic liquid crystalline phase carrier comprising one or more lipids forming the carrier and an antigen associated with the carrier.

2. The carrier of claim 1, wherein the one or more lipids is or comprises one or more amphiphilic lipids.

3. The carrier of claim 2, wherein the one or more amphiphilic lipids are selected from the group consisting of 1-monoolein, 2-monoolein, citrem, oleoyl lactate, oleamide, monoelaidin, linoleic acid, elaidic acid, monopalmitolein, monolinolein, phytantriol, diolein, triolein, dioleoyl-glycerol, 1,2-Dioleoyl-3-trimethylammonium propane (DOTAP), N—N-dioleoyl-N, N-dimethylammonium chloride (DODAC), dioctadecyl ammonium chloride (DOAC), dioctadecyl dimethyl ammonium chloride (DODMAC) or dioctadecyl dimethyl ammonium bromide (DODAB), 1,2-dioleoyl-sn-glycero-3-phosphoethanolamine (DOPE), 1,2-Dioleoyl-phosphatidylglycerol (DOPG), oleic acid (OA), lysol-hydroxy-2-oleoyl-sn-glycero-3-phosphocholine, 1,2-dioleoyl-sn-glycero-3-dihexyl-phosphocholine (DOPC), vitamin E tocopherol, vitamin E (tocopheryl) acetate, phytanoyl monoethanolamide, farnesoyl monoethanolamide, oleoyl monoethanolamide, linoleoyl monoethanolamide and linolenoyl monoethanolamide.

4. The carrier of claim 3, wherein the one or more amphiphilic lipids are monoolein (MO) and/or phytantriol.

5. The carrier of claim 3 or claim 4, wherein the carrier comprises MO or phytantriol in combination with one or more of cholesterol, DLPC, DSPC, DPPE, DPPS, DOPS, DPPC, DMPC, DMPS and DLPS.

6. The carrier of any one of the preceding claims, wherein the non-lamellar lyotropic liquid crystalline phase carrier is a cubosome or a hexosome.

7. The carrier of any one of the preceding claims, wherein the non-lamellar lyotropic liquid crystalline phase carrier has a particle size between about 10 micrometers and about 40 nanometers.

8. The carrier of any one of the preceding claims, wherein the non-lamellar lyotropic liquid crystalline phase carrier further comprises at least one stabiliser.

9. The carrier of claim 8, wherein the at least one stabiliser is one or more of a poloxamer, a surfactant or a PEGylated lipid.

10. The carrier of claim 8 or claim 9, wherein the at least one stabiliser is present at between 1 to 20 wt % of the carrier.

11. The carrier of any one of the preceding claims, wherein the antigen is encapsulated within the carrier.

12. The carrier of any one of the preceding claims, wherein the antigen is selected from the group consisting of a protein, a glycoprotein, a peptide, a glycopeptide, a polysaccharide, a lipid, a glycolipid, a lipoprotein, and a lipopeptide.

13. The carrier of any one of the preceding claims, wherein the antigen is hydrophobic or comprises at least one hydrophobic chain.

14. The carrier of claim 13, wherein the at least one hydrophobic chain is capable of being associated with or embedded in a lipid bilayer.

15. The carrier of any one of the preceding claims, wherein the antigen comprises one or more hydrophobic chains independently selected from fatty acids (lipids), glycerolipids, glycerophospholipids, sphingolipids, steroids, terpenes and terpeniods, saccharolipids, polyketides, and poly(hydrophobic amino acids).

16. The carrier of any one of the preceding claims, wherein the antigen is present at between about 0.1 to about 10 mol %, between about 0.1 to about 9 mol %, between about 0.1 to about 8 mol %, between about 0.1 to about 7 mol %, between about 0.1 to about 6 mol %, between about 0.1 to about 5 mol %, between about 0.1 to about 4 mol %, between about 0.1 to about 3 mol %, between about 0.1 to about 2 mol %, between about 0.5 to about 10 mol %, between about 0.5 to about 9 mol %, between about 0.5 to about 8 mol %, between about 0.5 to about 7 mol %, between about 0.5 to about 6 mol %, between about 0.5 to about 5 mol %, between about 0.5 to about 4 mol %, between about 0.5 to about 3 mol %, or between about 0.5 to about 2 mol %.

17. The carrier of any one of the preceding claims, wherein the antigen is cord factor trehalose 6,6′-dimycolate (TDM).

18. An immunogenic composition comprising a non-lamellar lyotropic liquid crystalline phase carrier of any one of claim 1 to claim 17 and a pharmaceutically acceptable carrier, diluent and/or excipient.

19. The immunogenic composition of claim 18, wherein the immunogenic composition is a vaccine composition.

20. The immunogenic composition of claim 18 or claim 19, wherein the immunogenic composition does not comprise or is substantially free of an adjuvant which is not a constituent part of the non-lamellar lyotropic liquid crystalline phase carrier.

21. A method of delivering an antigen to a cell including the step of contacting the cell with the non-lamellar lyotropic liquid crystalline phase carrier of any one of claim 1 to claim 17, or the immunogenic composition of any one of claim 18 to claim 20.

22. A method of inducing an immune response in a subject including the step of administering an effective amount of the non-lamellar lyotropic liquid crystalline phase carrier of any one of claim 1 to claim 17, or the immunogenic composition of any one of claim 18 to claim 20, to the subject.

23. A method of preventing, treating or ameliorating an infection, disease, disorder or condition including the step of administering a therapeutically effective amount of a non-lamellar lyotropic liquid crystalline phase carrier of any one of claim 1 to claim 17, or the immunogenic composition of any one of claim 18 to claim 20, to a subject in need thereof to thereby prevent, treat or ameliorate the infection, disease, disorder or condition.
